(19) United States
(12) Patent Application Publication
(10) Pub. No.: US 2011/0313407 A1
(43) Pub. Date: Dec. 22, 2011

(54) QUANTUM-DOT LASER DIODE

(76) Inventors: Edik U. Rafailov, Dundee (GB); Svetlana Zolotovskaya, Dundee (GB); Sergei Sokolovski, Dundee (GB)

(21) Appl. No.: 13/164,452

(22) Filed: Jun. 20, 2011

Related U.S. Application Data
(60) Provisional application No. 61/356,372, filed on Jun. 18, 2010.

(51) Int. Cl. A61B 18/20; B01J 19/12; H01S 5/323; B82Y 20/00

(52) U.S. Cl. 606/2; 372/45.01; 204/157.41; 977/773; 977/951

(57) ABSTRACT

Aspects of the present disclosure relate to the field of laser technology, specifically semiconductor lasers, and to novel biomedical applications of such lasers, including novel methods of photodynamic therapy. Exemplary embodiments of the present disclosure include a semiconductor laser diode having an active region having a gain medium with one or more InGaAs/InAs quantum dot layers; and wherein the laser diode can be arranged in operation to emit laser light having a central wavelength within spectral range of wave lengths. The present embodiments further include a method of directly forming a reactive oxygen species (ROS), the method including exposing a medium having a potential source of ROS to a semiconductor laser diode, the semiconductor laser diode configured to emit laser light having a central wavelength within the spectral range.

### RELATED APPLICATIONS

This application relates to U.S. provisional application No. 61/356,372, entitled “QUANTUM-DOT LASER DIODE,” filed on 18 Jun. 2010, and which is incorporated herein by reference in its entirety for all purposes.

### TECHNICAL FIELD OF THE INVENTION

The present invention relates to the field of laser technology, specifically semiconductor lasers, and to novel biomedical applications of such lasers, including novel methods of photodynamic therapy.

### BACKGROUND

Applications of Lasers in medicine have become widespread in the last decade. Lasers are frequently used in a multitude of different medical fields, ranging from ophthalmology, to oncology. In oncology, lasers are often used for photodynamic therapy (PDT). PDT itself relies on the photodynamic effect whereby photosensitised cells are damaged in the presence of light and oxygen. The light activation process of a PDT drug (P)(i.e. a photosensitizer) is initiated by the absorption of light to produce an exited singlet state (¹P*), which then populates a relatively long-lived triplet state (³P*) by intersystem crossing, P+hv→¹P*, ¹P→³P*. The longevity of the triplet state predominantly generates reactive oxygen species. As such, photosensitizer species existing in the excited triplet state react with oxygen in tissue, converting the oxygen molecule (O₂) into a reactive oxygen species. For example, converting O₂ from the normal triplet state (³Σ_{g}) form to a highly reactive excited singlet-state (¹Δ_{g}) form, ³P*+O₂(¹Σ_{g})→P+O₂(¹Δ_{g}). In turn, singlet oxygen can participate in free-radical chain reactions, oxidize amino acids in proteins or nucleotides in DNA, induce peroxide oxidation of lipids, and lead to apoptosis of malignant cells.

Conventional PDT methods require the presence of a photosensitizer species, as described above, to initiate the chain reaction of required chemical reactions, which terminates in the generation of highly reactive oxygen species (e.g. singlet oxygen). It is conventional in the prior art to use a laser as the light source for initiating the required chain reaction. The characteristics of lasers, such as the high degree of temporal and spatial coherence make them suitable for biomedical applications, such as PDT, where a spectrally selective action and the ability to make minor adjustments to the emitted laser beam (e.g. focusing, defocusing, coupling into a light delivery system) is required.

The therapeutic benefits of PDT derive from the activation of highly reactive oxygen species such as singlet oxygen molecules (in Type II photoreaction), which has a number of subcellular targets causing apoptosis of malignant cells, ultimately leading to cell necrosis. One commonly used PDT method used to treat internal anatomical regions requires ingestion by the patient of the photosensitizer species prior to treatment. PDT treatment subsequently follows, once the photosensitizer species has populated the anatomical region requiring treatment. This process may be time consuming, given that the photosensitising agent must first spread to the affected area intended for treatment, before the PDT treatment may be initiated. Alternatively, the photosensitizer species may be directly administered to the required anatomical region. This may be achieved by intravenous injection of the photosensitizer to the required anatomical region. For example, the photofrin (i.e. porfimer sodium) photosensitizer is commonly administered by intravenous bolus injection. For conditions such as skin cancer, the photosensitizer may be administered topically to the required site.

The effectiveness of the PDT treatment is often also dependent on the achievable concentration of photosensitising agent, which will vary depending on the anatomical region being targeted and the type of photosensitizer used. The suitability of current PDT methods is dependent on the physical location of the malignant cells, and will only be effective where it is possible to generate a sufficient concentration of photosensitising agent in order to be able to activate a sufficiently high concentration of reactive oxygen species.

Furthermore, due to the absorbance characteristics of skin tissue, which reduces the penetration depth of the activating light by absorption, often surgical intervention is required to treat internal tissue and organs. Therefore, known non-invasive (i.e. not requiring surgical intervention) uses of PDT are often restricted to the treatment of external tissue areas, or at best to tissue areas located only a few millimetres beneath the skin surface, due to absorbance of the stimulating light by the skin tissue.

The known applications and methods of PDT are limited by the above described issues, and although there have undoubtedly been successes with PDT, there is an urgent need for further research and improvements in phototherapy methods.

It is therefore an object of the present invention to overcome or substantially mitigate the above mentioned problems with the prior art. It is also an object of the present invention to provide a light source which is compact, and capable of generating the required optical power (i.e. light intensity) suitable for use in an improved PDT method which does not suffer the shortcomings of the prior art.

### SUMMARY OF THE INVENTION

One aspect of the present invention relates to a semiconductor laser diode comprising an active region having a gain medium comprising one or more InGaAs/InAs quantum dot layers. The laser diode is arranged in operation to emit laser light having a central wavelength within the spectral range of approximately 1250 to 1280 nm. More suitably, the central wavelength in within the range of approximately 1255 to 1275 nm or between approximately 1260 and 1270 nm.

One significant advantage associated with the semiconductor laser diode is that it emits laser light having a spectral bandwidth that falls within the so-called “therapeutic window”, where common tissue constituents demonstrate minimal absorption characteristics. Furthermore, the highest molecular oxygen energy absorption band also falls within the spectral range of laser light emitted by the laser. These characteristics of the semiconductor laser diode render it ideal for biomedical uses—for example, for use in photodynamic therapy.

A further advantage provided by the laser is its compactness—namely, its small physical size making it easily transportable—and the high output optical power achievable. These characteristics make the laser well suited for biomedical uses where mobility and high optical power output are required.

In a suitable embodiment of the present invention each InGaAs/InAs quantum dot layer comprises one InGaAs layer equal to one mono-layer and one InAs layer equal to two and a half monolayers. The InGaAs and InAs layers are arranged contiguous with each other, and wherein one or more quantum dots (QD) are comprised in the InAs layer.

Using an InGaAs/InAs quantum dot gain medium enables the laser to emit laser light having a central wavelength within the spectral range of approximately 1250 to 1280 nm. Furthermore, this is achieved whilst keeping the physical dimensions of the laser relatively compact, and easily transportable.

In alternative embodiments, however, the InAs quantum dot layer may comprise any suitable value between approximately 2 and 3 monolayers, such as between 2.2 and 2.8 monolayers, according to requirements.

The laser diode may further comprise a GaAs spacer layer arranged to separate adjacent InGaAs/InAs layers. The GaAs spacer layer may be arranged contiguous with the InAs layer defining a contact surface for facilitating the formation of an array of quantum dots in the InAs layer.

In preferred embodiments, the density of the quantum dots formed in the InAs layer is within the range of approximately 5×10¹⁰ to 6×10¹¹ cm⁻² per contact surface.

In certain embodiments, the number of layers of the InGaAs/InAs gain medium is at least three.

In alternative embodiments, the number of layers of the InGaAs/InAs gain medium is at least five.

In further alternative embodiments, the number of layers of the InGaAs/InAs gain medium is less than or equal to fifteen.

Each InGaAs/InAs gain medium layer is effectively a lasing source. Accordingly, the achievable output optical power may be increased by increasing the number of InGaAs/InAs layers comprised in the gain medium. A plurality of InGaAs/InAs layers may effectively be considered an array of laser sources.

In certain embodiments the full width at half maximum (FWHM) spectral bandwidth of the emitted laser light is less than approximately 50 nm. For example, the FWHM spectral bandwidth of the emitted laser light may be less than approximately 40 nm, or less than approximately 30 nm, and suitably less than approximately 25 nm. In more suitable embodiments, the FWHM spectral bandwidth of the emitted laser light is less than approximately 20 nm, or preferably less than approximately 12 nm.

The semiconductor laser diode may emit continuous wave laser light, or a pulsed laser light.

A second aspect of the present invention relates to a method of directly forming a reactive oxygen species (ROS). The method comprises exposing a medium comprising a potential source of ROS to a semiconductor laser diode, the semiconductor laser diode configured to emit laser light having a central wavelength within the spectral range of approximately 1250 to 1280 nm. More suitably, the central wavelength in within the range of approximately 1255 to 1275 nm or between approximately 1260 and 1270 nm.

Preferably, the medium comprising the potential source of ROS is substantially free of photosensitising agents that promote the formation of ROS. In preferred embodiments the methods of the invention are performed in the absence of any supplementary photosensitising agents.

Preferably, the medium comprises molecular oxygen.

In preferred embodiments the ROS is singlet oxygen.

An advantage associated with this aspect of the present invention is that a method for the direct activation of ROSs, such as singlet oxygen radicals, in the absence of a photosensitising agent is provided. The method may be in vivo or in vitro. In one embodiment, the method involves irradiating a medium, such as a fluid or tissue (e.g. in vitro, ex vivo or in vivo) with laser light emitted from the laser.

In preferred embodiments, the semiconductor laser diode used in the method comprises an active region having a gain medium comprising one or more InGaAs/InAs quantum dot layers.

Preferably, the semiconductor laser diode comprises two or more InGaAs/InAs quantum dot layers and a GaAs spacer layer arranged to separate adjacent InGaAs/InAs layers.

A third aspect of the present invention relates to a method for treating cancer in an individual. The method comprises exposing at least one tumour cell to laser light emitted from a semiconductor quantum dot laser diode at an intensity and for a period of time sufficient to generate reactive oxygen species (ROS) within or in the vicinity of the at least one tumour cell, whereby the death of one or more tumour cells occurs, and wherein the method does not involve the use of photosensitising agents that promote the formation of ROS within or in the vicinity of the at least one tumour cell.

In preferred embodiments singlet oxygen is generated from molecular oxygen within or in the vicinity of the one or more tumour cells, and wherein the emitted laser light has a central wavelength within the spectral range of approximately 1250 to 1280 nm. More suitably, the central wavelength in within the range of approximately 1255 to 1275 nm or between approximately 1260 and 1270 nm.

An advantage associated with this aspect of the invention, is that the semiconductor laser diode may be used for the activation of reactive oxygen species (ROS) in the vicinity of malignant cells. The ROS may suitably comprise singlet oxygen. The ROS (e.g. singlet oxygen) is suitably produced in a subject, such as a human body in the absence of a photosensitising agent by directly exciting oxygen species in the subject (e.g. triplet oxygen). The ROS may be produced in vivo or in vitro. The ROS may be produced in a tissue, such as the skin, or in or around a subcutaneous tissue or organ, such as a tumour cell/tumour.

For example, the laser may be used to irradiate a tumour or medium in close proximity to the tumour in vivo to activate singlet oxygen which subsequently reacts with neighbouring malignant cells. In some embodiments, necrosis or apoptosis of cells, such as malignant cells in the irradiated area or in the vicinity thereof may be caused by the ROS (e.g. activated singlet oxygen radicals). The ROS may, for example, react with proteins and/or nucleic acids in the (malignant) cells. Thus, the laser diode of the invention may have therapeutic applications, such as in the treatment of diseases and conditions in an animal (preferably a human), such as cancer.

Due to the penetrative tissue characteristics associated with the bandwidth of emitted laser light, the method of treatment may be non-invasive. In certain embodiments, ROS activation can occur without requiring any surgical intervention. For example, ROS (e.g. singlet oxygen) activation may be achieved within the human body for treatment of internally located malignant cells without requiring a photosensitising agent and without requiring surgical intervention.

Preferably, the semiconductor laser diode comprises two or more InGaAs/InAs quantum dot layers and a GaAs spacer layer arranged to separate adjacent InGaAs/InAs layers A number of further advantages over known prior art solutions are provided by the present invention, which are set out below in further detail in the description of the invention,

### BRIEF DESCRIPTION OF THE DRAWINGS

The invention will be further described with reference to the accompanying figures, in which:

FIG. 1 is a schematic illustration of a laser diode apparatus in accordance with the present invention.

FIG. 2 shows: (A) five different absorption spectra of oxygen (O₂) dissolved in various Freon type solvents registered at high pressure (130 atm), reprinted from Long & Kearns (1973), J. Chem. Phys. 59, pp 5729-5736); (B) a graph showing the absorption spectra of major tissue constituents and the emission wavelengths of common medical lasers; and (C) an example of the intensity profile, or equivalently the emission spectrum of laser light emitted from the laser diode apparatus of FIG. 1, when subjected to a forward bias of 5 A at an operating temperature of 30° C.

FIG. 3 is a schematic cross-sectional view of the gain medium used in the laser apparatus of FIG. 1.

FIG. 4 is a process flow chart outlining a method of photodynamic treatment using the apparatus of FIG. 1.

FIG. 5 shows the results of radiation-induced singlet oxygen production in anoxia air-saturated solutions. The graph shows the normalised concentration (y-axis) of air-saturated (half-filled circles); oxygen purified (open circles) and nitrogen purified (filled circles) solutions of naphthacene (115 μM) in CCl₄ after irradiation at approximately 1270 nm in the indicated dose (x-axis). Results were normalised against a control sample tested under normal room lighting conditions. The lines are a linear fit of the data from 3 experiments. Inset: absorption spectrum for naphacene In CCl₄. The main absorption maxima of naphthacene is located at 474 nm with no resolvable absorption in the vicinity of 1270 nm.

FIG. 6 illustrates ethidium fluorescence in HaCaT cells that have been irradiated to activate reactive oxygen species (ROS). Fluorescence recording time is indicated on the x-axis, signal strength is shown on the y-axis, and the period of time during which the cell medium was irradiated is indicated by the hashed block. The graph contains three traces from three different experiments, i.e.: the effect of NIR QD laser irradiation at approximately 47.7 J/cm² alone (dark grey triangle); the effect of NIR QD laser irradiation at approximately 47.7 J/cm² in the presence of the singlet oxygen scavenger, 10 μM α-tocopherol (grey circle); and the effect of 100 μM NaOCl in phosphate buffer saline (PBS) alone (black square).

FIG. 7 illustrates: (A) laser-induced dihydroxyethidium (DHOE) fluorescence in HaCaT cells (grey squares), primary keratinocytes (black triangles) and HeLa cells (dark grey circles) at an irradiation dose of approximately 47.7 J/cm²; and (B) laser-induced dihydroxyethidium (DHOE) fluorescence in HaCaT (black bars, left-hand column of each group), primary keratinocytes (dark grey bars, right-hand column of each group), and HeLa cells (light grey bars, middle column of each group at deferent irradiation doses. The results of a control experiment at a radiation wavelength of 830 nm (far right-hand group of columns) demonstrates that the observed results are not an artefact of cellular heating.

FIG. 8 shows the change in intracellular ‘free’ [Ca²⁺]ᵢ calcium ion concentration, as measured by fluorescence, in selected HaCaT cells induced by laser irradiation at a wavelength centred on approximately 1260 nm and an irradiation dose of 47.7 J/cm². The shaded area corresponds to the period of laser irradiation of the target cells (duration 3 min).

FIG. 9 shows the change in intracellular ‘free’ calcium concentration, [Ca²⁺]ᵢ as measured by fluorescence in HaCaT cells (grey square) and HeLa cells (dark grey circle) induced by irradiation at approximately 1260 to 1270 nm and a does of approximately 47.7 J/cm². A control irradiation dose of 47.7 J/cm² at approximately 830 nm is also shown (black triangle). The shaded area corresponds to the period of laser irradiation of the target cells (duration 3 min). The data is the mean result from 4 repeat experiments averaged over 5 to 15 cells±SE for each measurement.

FIG. 10 illustrates representative traces of single-channel ion currents recorded by the patch clamp method in HaCaT keratinocytes in cell-attached mode at a radiation dose of approximately 47.7 J/cm² (I) before, (II) during, and (III) after irradiation using a laser diode of the invention. Irradiation was performed at a wavelength centred on approximately 1260 to 1270 nm. The right-hand panel is a point-amplitude histogram (the vertical axis is scaled to traces) showing the results of single-channel event analysis by patch clamp technique: (I) before laser irradiation; (II) during a 3 min laser irradiation; and (III) after irradiation at a dose of 47.7 J/cm².

FIG. 11 illustrates the transmission spectrum of the electrophysiological bath solution used for patch clamp experiments.

FIG. 12 shows the temperature of the electrophysiological bath solution inside the measurement chamber as a result of irradiation at different doses for wavelengths of approximately 1260 to 1270 nm (grey squares) and 830 nm (grey diamonds). The lines are 3^{rd} order exponential fit of data from three experiments. Insert: detailed representative record of the temperature measurements with 1260 to 1270 nm and 830 nm lasers at 47.7 J/cm².

FIG. 13 depicts a representative trace of single-channel ion currents recorded by patch clamp technique in HaCaT keratinocytes in the presence of the ROS, e.g. singlet oxygen quencher (α-tocopherol, 10 μM) at an irradiation dose of approximately 47.7 J/cm².

FIG. 14 shows the results of a cell death assay using the CytoTox kit (Promega) measured 24 hours following different laser irradiation doses. Temperature increase in the measurement chambers did not exceed 6° C. from RT. BI2536, a PLK1 inhibitor that induces tumour cell death was used as a positive control.

### DETAILED DESCRIPTION

A more detailed description of specific embodiments of the present invention is set out below with reference to the above-described figures for illustrative purposes only.

The present invention relates to a novel laser and to a method of using the laser, to directly activate reactive oxygen species, such as singlet oxygen radicals by photoexcitation of oxygen molecules. In preferred embodiments the laser is a quantum dot semiconductor diode laser.

The present invention also relates to a method of PDT, wherein reactive oxygen species (ROS), and particularly singlet oxygen radicals, are directly activated in vivo for use in provoking biostimulative effects via singlet-oxygen mediated reactions. For example, the method of the present invention may be used for the therapeutic treatment of oncological and non-oncological conditions. The biomedical advantages of the present laser derive from the wavelength of the emitted laser light and the output power levels. As a result, the present laser may be used for non-invasive in vivo PDT. Of course, the laser and methods can also be used in vitro or for appropriate ex vivo applications.

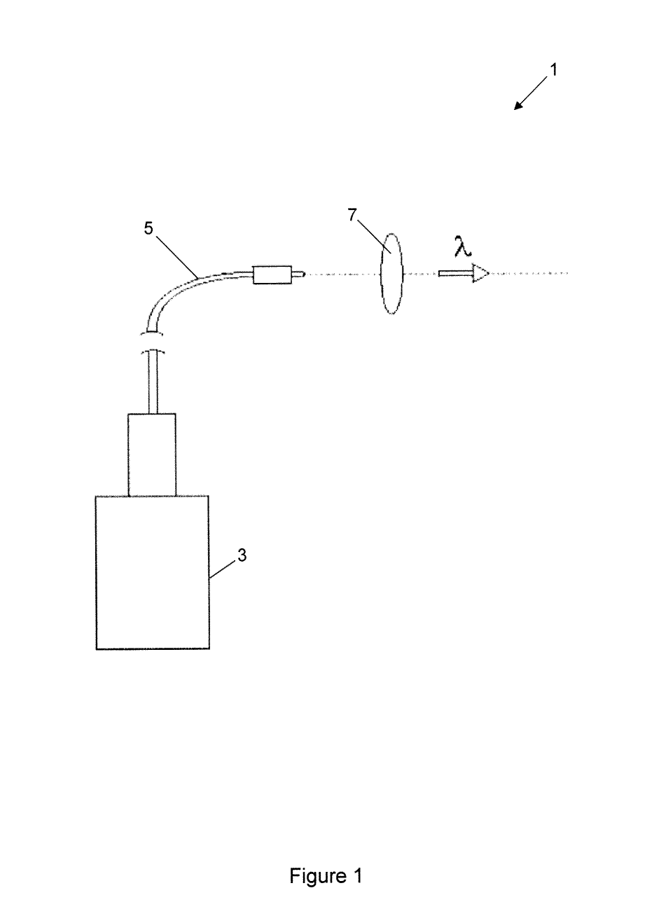

FIG. 1 is a schematic illustration of the laser apparatus 1, used to activate formation of reactive oxygen species, for example the activation of singlet oxygen radicals. The apparatus 1 comprises a continuous wave quantum dot semiconductor diode laser 3, along with an optical fibre 5 attached to the diode laser 3 for the accurate guidance of the output laser light to a selected target. The apparatus 1 may also comprise an optional lens 7. The lens is preferably a collimating (focusing) lens, optionally present to focus the emitted laser light to a smaller spot size having an increased incident power density. Preferably, the lens is fitted to the end of the optical fibre 5, and may be formed integral therewith. The optical fibre may be either single or multimode. In the following description of preferred embodiments the quantum dot semiconductor diode laser 3 of FIG. 1, may interchangeably be referred to as laser, semiconductor laser, diode laser, or quantum dot laser. It is to be appreciated that all references to a laser in the ensuing description of preferred embodiments are references to the quantum dot semiconductor diode laser 3 of FIG. 1.

In the description of the following preferred embodiments, a multimode optical fibre 5 is used, having a core diameter of 105 μm and a numerical aperture of 0.22. It is to be noted, that optical fibres with different physical characteristics (or any other light delivery systems) may equally be used for carrying out the below described methods. The optical fibre 5 facilitates the illumination of a user selected target area.

The gain medium of the laser diode is selected on the basis of the type of biostimulative reaction required. Specifically, the gain medium is selected on the basis of the required output laser light wavelength/frequency and output power. The principle use of the present semiconductor laser diode 3 is for direct activation (without requiring a photosensitising agent) of reactive oxygen species for example singlet oxygen, formed by photoexcitation of molecular oxygen (O₂), by irradiating the molecular oxygen with the output laser light as described previously.

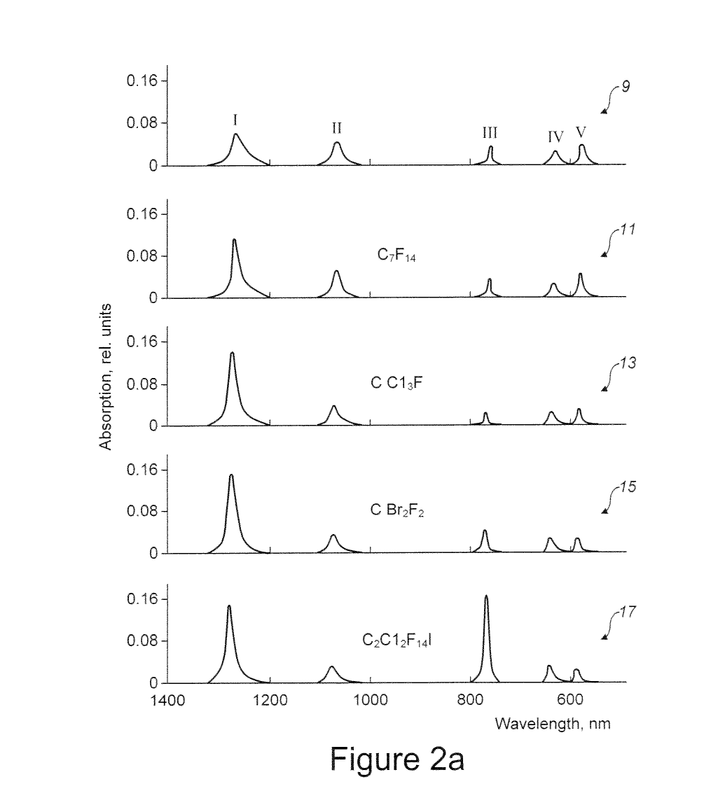

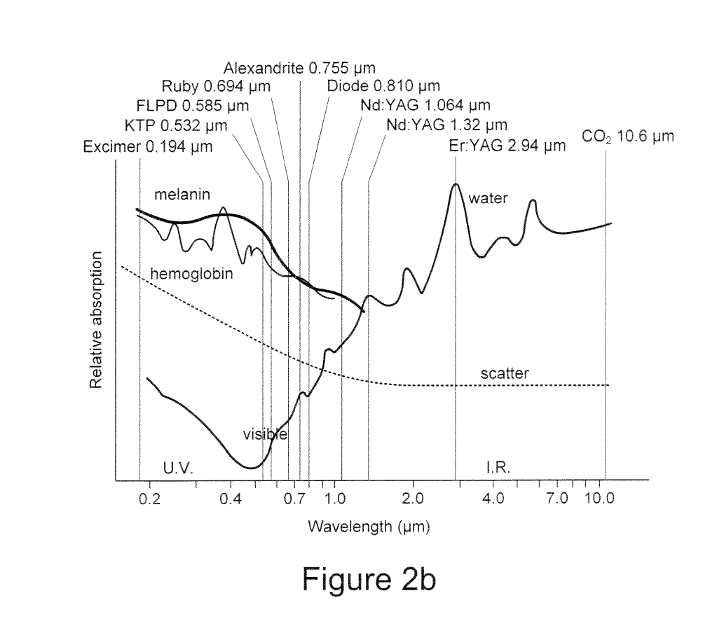

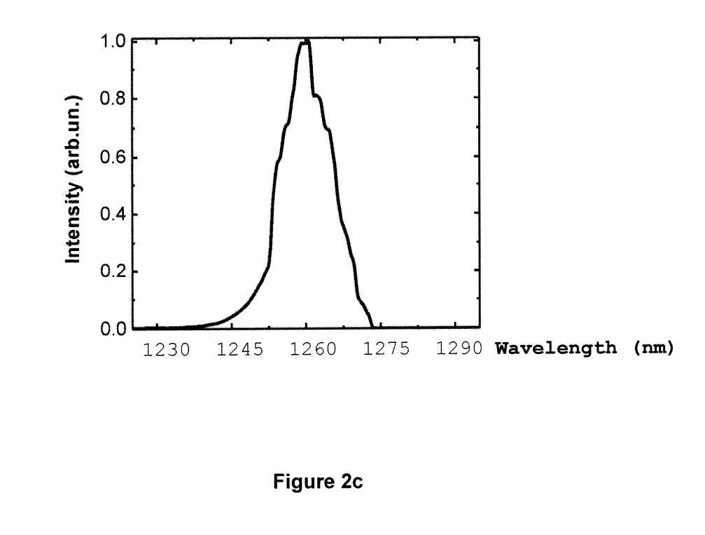

Before proceeding with the detailed description of preferred embodiments and by way of background, the skilled reader may appreciate that molecular oxygen (O₂) displays two distinct maximum absorption bands in the near infrared region—namely, at approximately 762 nm and at approximately 1270 nm. This is clearly observed from FIG. 2a, which is reproduced from Long & Kearns (1973) “Selection rules for the intermolecular enhancement of spin forbidden transitions in molecular oxygen,” J. Chem. Phys. 59, pp 5729-5736. FIG. 2a illustrates the high pressure absorption spectrum of O₂: in gas phase (9) and dissolved in various Freon-type solvents. A clear trend identified in the absorption spectra of FIG. 2a, is that O₂ displays five distinct electronic absorption bands (respectively labelled I to V) in the visible and near infrared (NIR) spectral region (e.g. from 500 nm to 1300 nm). The absorption bands are attributed to the transition of O₂ from the ground state ³Σ_{g-} to the singlet states ¹Δ_{g} when irradiated with optical radiation at corresponding wavelengths.

The first displayed absorption spectrum 9, is the high pressure absorption spectrum of O₂ in gas phase. The remaining absorption spectra 11, 13, 15, 17 were obtained by dissolving O₂ in different Freon-type solvents registered at high pressure (130 atm), and at approximately room temperature (23° C.). The Freon-type solvents included C₇F₁₄, CCl₃F, CBr₂F₂, and C₂Cl₂F₃I.

The different singlet state transitions are set out below with their corresponding peak positions:

³Σ_{g}_{^{−}}(O)+¹Δ_{g}(0)←³Σ_{g}_{^{−}}(O)+³Σ_{g}_{^{−}}(O) at 1270 nm I.

¹Δ_{g}(1)+³Σ_{g}_{^{−}}(O)←³Σ_{g}_{^{−}}(O)+³Σ_{g}_{^{−}}(O) at 1070 nm II.

¹Σ_{g}_{⁺}(O)←³Σ_{g}_{^{−}}(O) at 762 nm III.

¹Δ_{g}(0)+¹Δ_{g}(0)←³Σ_{g}_{^{−}}(O)+³Σ_{g}_{^{−}}(O) at 630 nm IV.

¹Δ_{g}(0)+¹Δ_{g}(1)←³Σ_{g}_{^{−}}(O)+³Σ_{g}_{^{−}}(O) at 580 nm V.

A complete treatment of the formation and/or activation of different radical oxygen species is beyond the scope of the present description. The interested reader is referred to any university level textbook on physical chemistry for a more complete discussion.

For present purposes it suffices to note that the intensity of absorption band I, which peaked at a wavelength of approximately 1270 nm, is the highest. This is clearly observed from the absorption spectra illustrated in FIG. 2a. It is also clear from FIG. 2a that direct molecular oxygen (O₂) activation is more effective in the near infrared (NIR) spectral range, at a wavelength of approximately 1270 nm.

FIG. 2b illustrates the absorption spectra of major tissue constituents, such as haemoglobin, melanin and water which are all commonly found in living tissue. In addition, the emission wavelengths of common medical lasers have been added to the graph. The “therapeutic window” is loosely defined as the spectral region where the major tissue constituents display minimal absorption—e.g. approximately in the region from 0.60 μm (600 nm) to 1.32 μm (1320 nm). A general observed trend is that the absorbance of the afore mentioned major tissue constituents decreases with increasing wavelength, whereas the absorbance of water increases with increased wavelength. Since water is present in most living organisms, and certainly in humans, this increasing absorbance of water with increasing wavelength must be taken into consideration when defining the “therapeutic window”. Specifically, the benefit derived from the decreasing absorbance of the major tissue constituents is counteracted by the increasing absorbance of water for increasing wavelength. Defining the “therapeutic window” as the spectral range of 600 nm to 1320 nm is a good approximation, where the benefits derived from the decreasing absorbance of the tissue constituents outweigh the increase in water absorbance. Accordingly, the absorption spectra of the major tissue constituents illustrated in FIG. 2b suggest that carrying out PDT in the “therapeutic window” offers the benefit of higher tissue penetration depth, accompanied with biostimulative effects. Furthermore, the wavelength of approximately 1270 nm (1.27 μm), which corresponds to a NIR absorption maxima of O₂, falls within this therapeutic window, as clearly seen in FIG. 2b.

Returning to the discussion of the laser apparatus of FIG. 1, the semiconductor laser 3 comprises a gain medium selected to output laser light having a wavelength bandwidth falling within the above-mentioned therapeutic region, and which also comprises the absorption maxima of O₂. In other words, the gain medium is selected such that the strongest absorption band of O₂—for example, 1270 nm—falls within the output laser light bandwidth. Preferably, the gain medium is selected such that the output laser light bandwidth is substantially centred with the strongest absorption wavelength of O₂. In this way, the laser light emitted by the semiconductor laser 3 may be used in PDT to activate reactive oxygen species, for example singlet oxygen radicals, from O₂ in the spectral region associated with minimum tissue absorption, and maximum tissue penetration depth.

In preferred embodiments, a gain medium of InGaAs/InAs is used in the semiconductor laser 3, which results in an output wavelength bandwidth ranging from 1250 nm to 1280 nm. Preferably, the bandwidth is substantially centred on 1260 nm. More preferably, the output bandwidth is centred on 1265 nm. The emitted laser light is characterised by having a bandwidth at full width half maximum (FWHM) of 12 nm, over the range 1250 nm to 1280 nm. It is important to note that the strongest absorption wavelength of O₂ falls within the emitted bandwidth of the laser 3.

As a result of the emitted wavelength bandwidth, activation of the reactive oxygen species, for example singlet oxygen radicals, occurs directly. Advantageously, the activation occurs in the absence of a photosensitising agent, by direct photoexcitation of O₂. As mentioned previously, direct photoexcitation is achieved by directly irradiating the molecular oxygen with the output laser light.

FIG. 2c is an example of the intensity profile, or equivalently the emission spectrum of laser light emitted from the quantum dot semiconductor diode laser 3 of FIG. 1, when subject to a forward bias of 5 A and an operating temperature of 30′C. The bandwidth of the illustrated intensity profile is substantially centred on 1260 to 1270 nm.

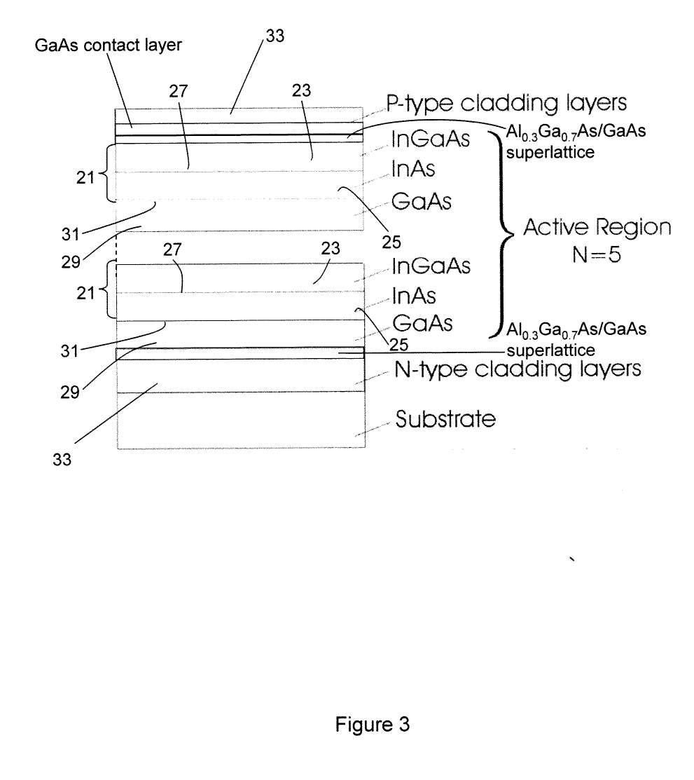

The semiconductor diode laser 3 comprises an InGaAs/InAs quantum dot gain medium, formed by one or more InGaAs/InAs layers, as illustrated in FIG. 3. Successive InGaAs/InAs layers are each separated by a GaAs spacer layer 29. Each InGaAs/InAs layer 21 is itself comprised of an InGaAs layer 23 arranged contiguous with an InAs layer 25, defining a first contact surface area 27. Each InAs layer is also arranged contiguous with the GaAs spacer layer 29, to define a second contact surface area 31 arranged opposite to the first contact surface area 27. Effectively, the InAs layer 25 is sandwiched between a InGaAs layer 23 and the GaAs layer 29.

The second contact surface area 31, located between the InAs layer 25 and the GaAs spacer layer 29 facilitates the formation of an array of quantum-sized three-dimensional islands on the GaAs layer 29 during manufacture of the gain medium. In this way, the InAs layer 25 is populated with a plurality of quantum dots. Further information regarding the formation and growth of the InGaAs/InAs layer 21 is set out in the ensuing paragraphs.

Preferably, the gain medium comprises a plurality of InGaAs/InAs layers 21, each separated from its neighbouring layer by the GaAs spacer layer 29. Each InGaAs/InAs layer 21 may be viewed as a lasing source, emitting light when subjected to an applied bias, which is in phase with the optical light emitted by neighbouring InGaAs/InAs layers 21, resulting in constructive interference. In other words, the intensity of the light emitted from one InGaAs/InAs layer is amplified by the light emitted by neighbouring InGaAs/InAs layers. Multiple InGaAs/InAs layers effectively form an array of lasing sources.

The mechanics of semiconductor lasing are well known in the technical field and accordingly a description of the semiconductor lasing action is not included herein. Instead, the interested reader is referred to the textbook titled “Quantum Dot Laser” (Oxford University Press, 2003) by V. M. Ustinov, et al. for a complete treatment of the mechanics of semiconductor lasing.

FIG. 3 is a schematic cross-section of a multi-layer InGaAs/InAs gain medium for use in the semiconductor laser 3. Each InAs layer 25 is a quantum dot gain structure, which may be grown on an N+ GaAs(100) substrate by molecular beam epitaxy. For example the gain medium may be grown, and quantum dot islands formed using the Stranski-Krastanov growth. The skilled reader will appreciate that substrate temperature, arsenic pressure, growth rate in number of monolayers per second (ML/sec) and indium monolayer coverage (ML coverage) are all significant parameters in Stranski-Krastanov growth. During manufacture, the indium coverage is controlled by the opening time of the indium source shutter, a longer shutter time results in higher ML coverage, which results in a higher quantum dot density. The thickness of the InAs layer is calculated as the used growth rate (ML/sec) multiplied by the opening time of the indium source shutter (sec), and as a result the deposited material thickness will be in monolayers (ML).

In preferred embodiments the InAs layer is a 2.5 mono-layer. Each layer of quantum dot islands is formed by deposition of 2.5 mono-layers of InAs followed by a 5.5 nm thick InGaAs quantum well. In preferred embodiments, the density of InAs quantum dots present on each GaAs layer is suitably in the range of approximately 1×10¹⁰ to 1×10¹² cm⁻²; such as approximately 5×10¹⁰ to 6×10¹¹ cm⁻². Preferably, the density is of the order of 10¹⁰ cm⁻², for example, approximately 5×10¹⁰ cm⁻². The linear dimensions of the quantum dots can vary, but are typically in the range of 5×5×5 nm to 15×15×15 nm. For example, the quantum dot sizes may suitably be in the order of approximately 7×12×12 nm.

The gain medium further comprises N- and P-type Al_{0.8}Ga_{0.2} As cladding layers 33, which help to conduct charge through the gain medium to initiate lasing, when the gain medium is subjected to an applied bias. The cladding layers are doped with Si and Be respectively, with a carrier concentration approximately in the region of 5×10¹⁷ cm⁻³ to 10¹⁹ cm⁻³. Preferably, the carrier concentration is in the range up to approximately 10¹⁹ cm⁻³; for example, between approximately 10¹⁶ and 5×10¹⁶ cm⁻³, or between approximately 5×10¹⁶ and 10¹⁸ cm⁻³. Suitably the carrier concentration is approximately 5×10¹⁷ cm³. The multi-layer InGaAs/InAs gain medium (i.e. the active region), including the GaAs spacer layers are placed in the centre of a short-period Al_{0.3}Ga_{0.7} As (2 nm)/GaAs (2 nm) superlattice, which acts as a waveguiding layer. The total thickness of the laser waveguide (comprised of the InGaAs/InAs layer, GaAs spacer layer and superlattice structure) is in the range of 0.25 to 0.5 μm, such as approximately 0.35 μm (350 nm). The gain structure is completed by an approximately 0.6 μm (600 nm) thick GaAs contact layer that is highly doped with Be. It is to be noted that this GaAs contact layer is distinct from the GaAs spacer layer 29. The function of the contact layer is to facilitate the passage of charge through the gain medium for the purposes of lasing.

In a preferred embodiment, the laser active region consists of five InGaAs/InAs layers 21, each layer comprising a layer of islands of self-organised quantum dots. Each InGaAs/InAs layer 21 is separated by a 33 nm thick GaAs spacer layer 29. Using this gain medium in the semiconductor laser 3 of FIG. 1, irradiation doses between 6 and 600 Jcm⁻² are achievable with an applied forward bias of up to 7 A and exposure times of up to 10 minutes. The same range of irradiation doses may be achievable with lower forward biases, provided that exposure time is accordingly increased.

A further aspect of the invention, relates to a method for directly activating reactive oxygen species, for example singlet oxygen radicals. In particular, the emission characteristics of the present quantum dot semiconductor diode laser 3 may be used to directly activate reactive oxygen species, for example singlet oxygen radicals by photo-excitation of O₂ in vivo. For example, the present method may be used for in vivo PDT in oncology. For example, the in vivo PDT treatment of malignant cells and/or tumours.

Tumours are comprised of a plurality of malignant cells, which comprise oxygen molecules. Additional oxygen molecules will be present within the vicinity of malignant cells in vivo. Activation of the reactive oxygen species, for example singlet oxygen radicals, occurs through photo-stimulation of the oxygen molecules, using the above-described semiconductor diode laser 3. The activated reactive oxygen species subsequently react with neighbouring malignant cells, thereby provoking apoptosis and/or necrosis of the malignant cells. Apoptosis prevents the further growth of tumours, and will result in a reduction in size of the tumour, which eventually leads to the destruction of the tumour.

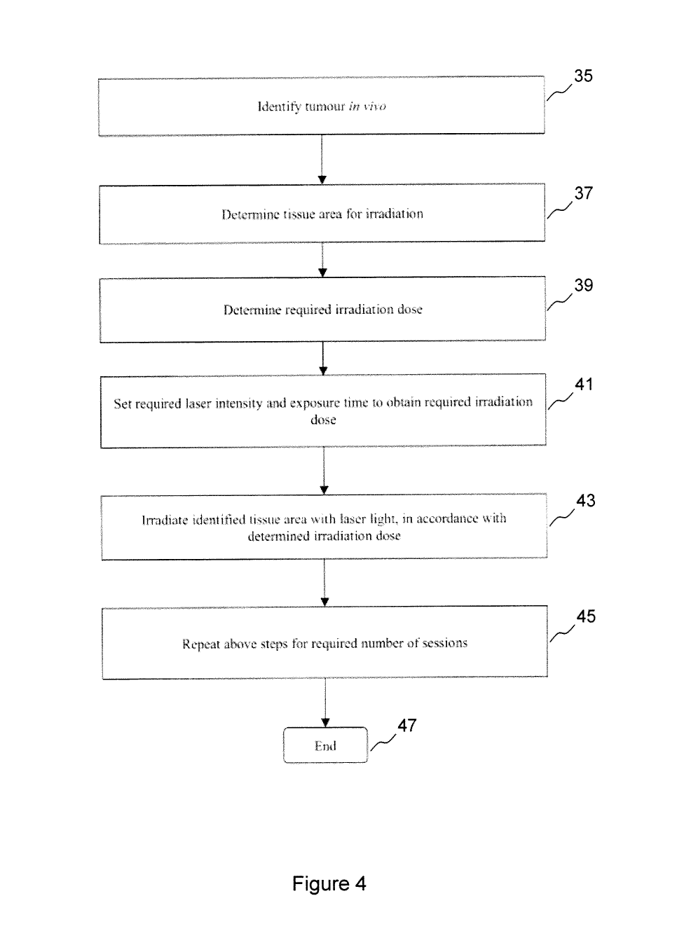

FIG. 4 is a process flow chart providing an example of a method of in vivo PDT treatment of tumours, using the semiconductor laser 3 of FIG. 1. The method is initiated by determining the location of the tumour at step 35. Identification of tumours and malignant cells may be achieved using conventional techniques, familiar to the skilled reader, and falling outside the scope of the present invention. Magnetic resonance imaging (MRI) is one example of a conventional method used for the identification of tumours and malignant cells. Further alternative identification methods may also be used in conjunction with the present method of PDT. It is envisaged that any existing diagnostic method may be used with the present method, provided that the diagnostic method provides information regarding the location of the malignant cells and/or tumours in vivo. Since such diagnostic methods are well known in the art, no further discussion of the methods is enclosed herein.

Once the location of malignant cells has been identified, it is necessary to determine the tissue area which will be irradiated at step 37. As a precautionary measure, the determined irradiation area may be larger than the determined tumour area. For example, cells and/or tissue area neighbouring the identified tumour may also be irradiated to minimise the likelihood of any tumour cells surviving the PDT treatment. The objective of the current PDT method is to provoke apoptosis and/or necrosis of the malignant cells and/or tumour cells. In step 39 the required irradiation dose is determined on the basis of the location of the malignant cells. For example, the depth of the malignant cells and/or tumour will affect the required irradiation dose. Equally, the location of the malignant cells and/or tumour, and in particular the tissue surrounding the cells and/or tumour may be taken into consideration when determining the required irradiation dose. Once the required irradiation dose has been determined, both the required laser intensity, or equivalently the required output optical power, and the exposure time may be determined, since both are functions of the required irradiation dose. The skilled reader will appreciate that irradiation dose is effectively determined by multiplying the applied optical intensity by the exposure time, and has the units of J/cm². It is to be noted that the required irradiation dose, output optical intensity, and exposure time are all selected on the basis of the location of the malignant cells, the size of the area affected by the malignant cells and the density of the malignant cells within the affected area.

The optical power output of the laser diode is variable and is dependent on the bias applied across the gain medium—in general the higher the applied bias, the greater the output optical power and intensity. In step 41, the different laser parameters are set. For example, the required output laser light intensity and the exposure time are determined on the basis of the irradiation dose determined in step 39. The laser light intensity is set by selecting the bias applied across the gain medium. For example, and as stated previously irradiation doses between 6 and 600 Jcm⁻² are achievable, with an applied forward bias of up to 7 A and exposure times of up to 10 minutes. The skilled reader will appreciate that for a given irradiation dose, output laser light intensity and exposure time are inversely proportional. For example, the same irradiation dose may be obtained at a lower output laser light intensity, by proportionally increasing the exposure time. Similarly, the same irradiation dose may be maintained by decreasing exposure time, provided this is accompanied by a corresponding increase in output laser light intensity.

Once the laser parameters have been set in step 41, the malignant cells are directly irradiated in step 43 with the output laser light having a wavelength bandwidth centred on between approximately 1260 and 1270 nm. For example, the wavelength of emitted light may be centred on any wavelength within approximately this range, such as approximately 1260 nm, approximately 1265 nm or approximately 1270 nm. As mentioned previously, a significant benefit of this output wavelength is that a high degree of tissue penetration is achievable. In this way, the present method provides a means for directly irradiating, and photo-exciting oxygen molecules existing within a selected in vivo area. This comprises photo-exciting oxygen molecules present within and adjacent to malignant cells lying beneath skin tissue, in a non-invasive manner—surgical intervention is not required due to the penetrative characteristics of the output laser light operating in the “therapeutic window”. This feature of the present PDT method, provides a significant advantage over existing PDT solutions.

Existing PDT solutions use optical radiation having wavelengths which are more readily absorbed by skin tissue and also require a photosensitising agent to activate the required reactive oxygen species. This is in part due to the inherent misconception in the prior art that a photosensitising agent is essential to activate the required reactive oxygen species, and to the fact that optical radiation sources emitting optical radiation having a bandwidth substantially corresponding with the absorbance maxima of molecular oxygen are not known. The direct activation of reactive oxygen species, for example singlet oxygen, in the absence of a photosensitising agent is not achievable by known solutions. As a result of the lack of tissue penetration achieved with known PDT methods, surgical intervention is often required for treatment of deeper lying malignant cells.

Returning to the discussion of FIG. 4, irradiating the target area with the required intensity of laser light, for the determined exposure time, causes activation and formation of reactive oxygen species, for example the activation singlet oxygen radicals, through direct photoexcitation of the oxygen molecules present in the target area as described previously. This includes, oxygen molecules present within the malignant cells and in the vicinity of malignant cells. The activated reactive oxygen species, for example the singlet oxygen radicals, are extremely reactive and unstable, and react with the malignant cells, causing apoptosis of the cells and/or necrosis.

Further photodynamic treatment sessions may be required, depending on the number and density of the malignant cells in the target area, until all malignant cells have undergone apoptosis and/or necrosis. Accordingly, in step 45 if it is determined that further treatment sessions are required then steps 35 to 43 may be repeated for the required number of sessions, until the tumour and/or malignant cells have undergone apoptosis and/or necrosis. Once the tumour cells have undergone apoptosis and/or necrosis, the method is ended in step 47.

Although the above described embodiments have been described with respect to specific quantum dot sizes and densities, it is important to note that other densities, and sizes of quantum dots are also envisaged in the semiconductor diode laser used in carrying out the afore described method of directly activating reactive oxygen species and the PDT method.

It is also to be noted that the number of InGaAs/InAs gain medium layers used is dependent on the required output optical power required from the semiconductor diode laser, and it is envisaged that for some applications up to thirteen layers or more may be required.

### Lasers in Biomedical Applications

The special feature of the laser radiation is its high degree of monochromaticity, allowing spectrally selective action on biomolecules, well matched with their absorption and, consequently, to the light-action spectra. Since the first demonstration of laser emission at a wavelength of 694 nm (Maiman (1960), Nature, 187, pp 493-494), the available wavelength domain of biomedical laser sources has been significantly broadened, ranging from far infrared to far ultraviolet (Peng et al., (2008), Rep. Prog. Phys., 71, 056701, pp 28).

Probably the most fundamental difference between laser emission and radiation from other light sources is that lasers have the potential for generating beams with very high spatial coherence, e.g. Gaussian beams. As a result, such beams exhibit good focusability and the potential to form collimated beams with very low beam divergence. A laser can therefore be focused to a spot with a diameter of only a few microns so that it can irradiate intracellular structures; but can also be defocused to cover as big an application area as required. Although a high degree of time and spatial coherence can also be obtained from fluorescent tubes, discharge and incandescent lamps by spectral and spatial filtering of the emission, the spectral radiance of laser sources, i.e. emitted power per unit area of the beam cross-section, per unit of open solid angle and unit wavelength, still remains unachievable for all other light sources. Further, the ability to generate ultra-short pulses in the region of several femtoseconds (Ell, et al., (2001), Opt. Lett. 26, pp 373-375) is a unique feature of laser sources which enables them to supply irradiation intensity of the order 10¹¹⁻¹² W/cm². The combination of these unique capabilities, absent in any other type of radiation source, makes lasers a universal and efficient biomedical tool (Waynant, ed., Lasers in medicine, CRC Press, 2002; Berlien & Miiller, eds., Applied laser medicine, Springer-Verlag, 2003; and Goldman, ed., The biomedical laser, Springer-Verlag, 1981).

Semiconductor laser diodes—due to the advantages of compactness, efficiency, reliability, direct electrical control, and a facility for direct integration with optical fibres—will indisputably lead to a silent revolution in biomedical applications. Recent progress in semiconductor material science and technology has enabled the extension of the available range of coherent light sources within the spectral range, known as the therapeutic (or diagnostic) window, ranging from 600 nm to 1.3 μm. Within this range, we have found that most tissues exhibit weak absorption, which permits a significant penetration depth of the laser radiation, as described elsewhere herein.

Laser radiation interacts with living matter at various biological organisation levels such as the biomolecule, subcellular structures, biotissues, organs and the whole organism. All types of laser-biomatter interactions can be categorised as resonance processes, involving absorption of the light with the resultant excitation of biomolecules, and non-resonance processes, arising from scattering of laser radiation by molecular vibrations and refractive index irregularities in the biotissue exposed, or its reflection from boundaries between media with different refractive indices. When a biomolecule absorbs light, the expenditure of the excitation energy can flow along various pathways, depending on the type of excitation of the molecules, i.e. electronic or vibrational, its surroundings, and radiation intensity. Some absorbed energy degrades to heat in a radiation-less manner, e.g. vibrational excitation by infrared light degrades to heat in a picosecond timescale, and some of it may be transferred to the neighbouring molecules. The electronically exited molecules may take part in chemical reactions or they may be excited to higher quantum states and then be involved in photochemical processes. Almost all these excitation pathways may be successfully utilised in laser biomedicine: laser fluorescence diagnostics, laser photodynamic therapy, laser phototherapy and laser thermal surgery (Vo-Dinh, ed., Biomedicalphotonics, CRC Press, 2003; Waynant, ed., Lasers in medicine, CRC Press, 2002; Berlien & Miiller, eds., Applied laser medicine, Springer-Verlag, 2003; and Goldman, ed., The biomedical laser, Springer-Verlag, 1981). As was noted, laser radiation parameters, i.e. wavelength, spectral bandwidth, intensity and pulse duration, can be varied over a wide range, thus making it possible to implement various types of light-biomatter interaction, i.e. linear and non-linear, single- and multiple-photon, coherent and non-coherent, thermal and non-thermal, etc. Thereby, one can induce various effects in biotissue, such as photochemical modification, thermal destruction, explosive ablation, optical breakdown, shock pressure waves, photo-disruption, etc.

Fluence rates below hyperthermia level are widely implemented in the laser therapy methods, which can be subdivided into two classes: low-intensity laser therapy and photodynamic laser therapy. Low-intensity laser therapy is based on the excitation of endogenous chromophores in biotissue, i.e. nucleotides, amino acids, water, protein-specific chromophores, etc., following a chemical reaction in the excited state of cellular constituents. The mechanisms of photochemical reaction are varied, including photo-addition, photo-fragmentation, photo-oxidation, photo-hydration, photo-isomerisation and photo-rearrangement (Prasad, Introduction to biophotonics, Willey, 2003). Although the primary mechanism of light action after absorption of light quanta and the promotion of electronically excited states has not yet been established, there have been several hypotheses on the possible primary reactions in photo-acceptor molecules made to date.

Historically, the first mechanism proposed was so called “singlet-oxygen hypothesis” (Karu et al., (1981), Lett. Nuov. dm. 32, pp 55). Certain photo-absorbing molecules, like porphyrins and flavoproteins (some respiratory-chain components belong to these compound classes), can be reversibly converted to photosensitizers (Karu, et al., (1982), Nuov. dm et al. D 1, pp 828-840). Based on visible laser light action on RNA synthesis rates in HeLa cells and spectroscopic data for porphyrins and flavins, the hypothesis was put forward that the absorption of light quanta by such molecules was responsible for the generation of singlet oxygen ¹O₂ and, therefore, for stimulation of the RNA and DNA synthesis rates (Karu et al., (1981); and Karu et al., (1982). This possibility has been considered as a dominant suppressive reaction when cells are irradiated at higher doses and intensities.

Another mechanism that has been proposed relates to the changes in redox properties and acceleration of electron transfer, and is known as “redox properties alteration hypothesis” (Karu, (1988), Lasers Life Sci., 2, pp 53-74). Photoexcitation of certain chromophores in the cytochrome c oxidase molecule, like Cu_{A} and Cu_{B} or hemes a and a₃, influences the redox state of these centres and, consequently, the rate of electron flow in the molecule.

The respiratory chain activation by light irradiation that leads to increased production of superoxide anions has been considered and is regarded as the “superoxide anion hypothesis” (Karu et al., (1993), Lasers Surg. Med. 13, pp 453-462). It has been shown that the production of O₂′⁻ depends primarily on the metabolic state of the mitochondria. Some recent developments indicate that under certain physiological conditions the activity of cytochrome c oxidase is also regulated by nitric oxide (NO; Brown, (1999), Biochem. Biophys. Acta 1411, pp 351-369). This regulation occurs via reversible inhibition of mitochondrial respiration. The “NO hypothesis” assumes that laser irradiation and activation of electron flow in the molecule of cytochrome c oxidase could reverse the partial inhibition of the catalytic centre by NO, and in this way increase the O₂-binding and respiration rate. Recent experimental results on the modification of irradiation effects with NO donors do not exclude this hypothesis (Karu et al., (2001), Toxicol. Lett. 121, pp 57-61).

The “transient local heating hypothesis” observes that a noticeable fraction of the excitation energy is converted to heat, which causes a local transient in the temperature of absorbing chromophores, leading to structural changes and may trigger biochemical activity, e.g. cellular signalling or secondary dark reactions (Karu et al., (1991), J. Photochem. Photobiol., B 10, pp 339-344).

Even though the effectiveness of treatment by low-intensity laser therapy has been proven by a number of clinical trials (Turner & Hode (1999), Low level laser therapy—Clinical practice and scientific background, Prima Books), the question on which mechanism is decisive in biostimulative effects at low irradiation intensities still remains open.

The prior art in photodynamic therapy (PDT) requires the presence of exogenous chromophores that perform the function of photo-sensitisation, with photo-addition and photo-oxidation as dominant mechanisms (Prasad, Introduction to biophotonics, Willey, 2003). Lesion-localised oxidative damage inflicted by photodynamic processes is initiated by the light activation process of a photosensitizer (^{o}S) to produce an exited singlet state ¹S*), which then populates a relatively long-lived triplet state (³S*) by intersystem crossing, following generation of the reactive oxygen species via two different pathways, designated as type I and type II. A type I process generates reactive free radicals, peroxides, and superoxides by electron or hydrogen transfer reaction with water or with biomolecule to produce a cytotoxic effect. In a type II pathway, however, the excited triplet state of the photosensitizer reacts with the oxygen in the tissue and converts the oxygen molecule from the normal triplet state (³Σ_{g}−) to a highly reactive excited singlet state ¹Δ_{g}. Singlet oxygen can participate in free-radical chain reactions, oxidise amino acids in proteins or nucleotides in DNA, induce peroxide oxidation of lipids, and may ultimately lead to the destruction of malignant cells. It is the type II process that is generally accepted as the major pathway for PDT (Vo-Dinh, ed., (2003), Biomedicalphotonics, CRC Press).

Although PDT treatment is gaining widespread clinical application in both oncologic and non-oncological applications, there is an urgent need for further research and improvements in phototherapy methods. For example, in all the prior art cases, a photosensitizer is essential to the performance of the PDT treatment. While the present invention is not to be considered limited by theory or by any proposed mechanism of action, it is notable that in some methods of the invention a type II pathway may be particularly beneficial. However, preferably, the methods of the invention are performed in the absence of any recognised (e.g. added/exogenous) photosensitising agent. Thus, the methods of the invention may be easier to perform, cheaper, involve fewer and potentially less toxic additional chemicals, and may be less invasive than the prior art methods.

Thus, according to the present invention, one particular opportunity involves activation of the biological system response through “direct” activation/formation of reactive oxygen species (ROS) without the requirement for a photosensitizer. Most suitably, the methods of the invention activate molecular oxygen (to singlet oxygen) by direct photo-excitation. By “direct” in relation to activation/formation of ROS and/or singlet oxygen, it is meant that the excitation light/energy acts on the ROS or singlet oxygen precursor itself in order to cause said activation or formation. Thus, the process does not require supplementary or intermediate compounds for transmitting the incident energy to the oxygen species. This is in contrast to the “indirect” excitation processes of the prior art, which act via and require a photosensitising agent. In other words, where a photosensitising agent is excited by the incident light/energy to a higher excitation level and the photosensitizer then passes its energy onto an oxygen species to create an ROS (as per the recognised prior art), such a process is considered herein to be an indirect mechanism for activating an ROS.

The feasibility of singlet molecular oxygen photo-activation in the absence of photo-sensitising agent remains highly debatable due to the fact that the direct O₂ (¹ Δ_{g})←O₂ (³Σ_{g}⁻) transition appears to be doubly forbidden on the basis of the spin-orbital selection rules. However the action spectra of a number of cell cultures recorded in the spectral range from 310 to 860 nm, and the results for low-intensity laser therapy, suggest a transformation of cell metabolism in response to low power laser excitation in the spectral intervals consistent with absorption bands of oxygen, (Waynant, ed., Lasers in medicine, CRC Press, 2002), O₂ (¹Δ_{g}); and formation by photo-excitation in the near infrared spectral range have also been demonstrated in pigment-free aerobic systems (Krasnovsky, et al., (2003), Biochem. 68, pp 1178-1182; and Krasnovsky & Ambartsumian (2004), Chem. Phys. Lett. 400, pp 531-535).

In particular embodiments, this invention is directed towards the direct activation of ROS in sources/mediums comprising suitable oxygen-containing species. Advantageously, the invention is directed towards the direct activation of single oxygen in organic solutions and/or mediums of singlet oxygen traps. The medium concerned may comprise living cellular systems, such as individual cells, cultures, tissues or organisms.

Although the energy for activation may be derived from any suitable radiation source, in some embodiments the use of an InGaAs/InAs quantum dot based laser diode emitting in the near infrared spectral range is particularly suitable. The results on photo-activation of molecular oxygen in air-saturated solutions of singlet oxygen traps are reported in the Examples. Laser-induced single channel activation in HaCaT immortalised skin keratinocytes is also presented in the Examples; along with the fluorescence analysis of HaCaT skin keratinocytes cells by means of the exogenous reporter molecules in response to the laser irradiation.

### Reactive Oxygen Species

The first observation of biological substrate photo-oxygenation in the presence of fluorescence dyes with the action spectra matched to the absorption spectra of the dyes (Tappeiner & Jodlbauer (1904), Deut. Arch. Klin. Med. 80, pp 427-487), has initiated intensive research on the nature of photodynamic action and its possible pathways in living cells, tissues and the whole ecosystem. This observation, accompanied with some early suggestions on the role of peroxides in activation of the oxygen (Straub (1904), Arch. Exp. Pathol. Pharmacol., 51, pp 383-390) and the discovery of singlet oxygen and its free radical derivatives (Kasha & Frimer ed., Singlet O₂, CPC Press, 1985, vol. 1, pp 1-11), has led to the establishment of the primary mechanisms of the photodynamic action.

According to the molecular-orbital theory, the outermost two electrons of O₂ in the ground state ³Σ_{g}− occupy different π*2p antibonding orbitals with parallel spin (t,t). Molecular oxygen's lowest excited singlet state is represented by two configurations: the ¹Σ_{g}+ state, with paired electrons in the same n* orbitals as in the ground state but with opposite spin (↑,↓), and the ¹Δg state, with electrons paired in one orbital (↑↓). As in both forms the spin restriction is removed, the oxidizing ability of O₂ is greatly increased. The ¹Δ g O₂ state has energy of 93.6 kJ above the ground state. The ¹Σ_{g}+ O₂ state is even more reactive with energy 157 kJ above the ground state. The superoxide radical, O₂²^{_{•}}⁻, has one unpaired electron which enters one of the n*2p antibonding orbitals. A non-radical O₂ derivative is represented by peroxide ion, O₂²⁻. Since the extra electrons in O₂²^{_{•}}⁻, and O₂²⁻ are located in antibonding orbitals, the strength of the O—O bond drops. Addition of two more electrons to O₂²⁻ eliminates the bond entirely since they are allocated into the π*2p orbitals, giving two oxide ions, O²⁻In biology the two-electron reduction product of O₂ is hydrogen peroxide (H₂O₂) and the four-electron product is water:

O₂→two-electron reduction (plus 2H⁺)→H₂O₂ (protonated form of O₂²⁻)

O₂→four-electron reduction (plus 4H⁺)→2H₂O (protonated form of O²⁻)

Oxygen radicals are usually referred to as reactive oxygen species (ROS), though this is a collective term which includes some non-radical derivatives of O₂, such as H₂O₂, hypochlorous acid (HOCl), and ozone (O₃) (Halliwell & Gutteridge, Free radicals in biology and medicine, Oxford University Press, 2007), as listed below.

### Reactive Oxygen Species (ROS):

| Radicals | Non-radicals |
|---|---|
| Superoxide, O₂²•⁻ | Hydrogen peroxide, H₂O₂ |
| Hydroperoxyl, HO₂²• | Peroxynitrite, ONOO⁻ |
| Hydroxyl, OH• | Peroxynitrous acid, ONOOH |
| Peroxyl, RO₂• | Nitrosoperoxycarbonate, ONOOCO₂⁻ |
| Alkoxyl, RO• | Hypochlorous acid, HOCl |
| Carbonate, CO₃•⁻ | Hypobromous acid, HOBr |
| Carbon dioxide, CO₂•⁻ | Ozone, O₃ |
| Singlet O₂ (¹Σ_{g}+) | Singlet O₂ (¹Δ_{g}) |

The production and/or activation of any of the above ROS are encompassed within the methods of the present invention. Any such ROS, when produced by the methods and/or using the apparatus of the invention may lead to the death of target cells, such as tumour cells, which may be thus be used in the treatment of diseases such as cancer. A preferred ROS activated in accordance with the invention is singlet oxygen, which may be produced by activation of molecular oxygen.

In accordance with the invention, the ROS (or singlet oxygen) may be activated within a target tissue or cell, or in the medium surrounding (i.e. in the vicinity) of the target tissue or cell. Thus, the relevant ROS may be directly produced inside one or more cells, e.g. tumour cells, or adjacent to the target cells such that they interact with the outside of the cells and/or may be transported or otherwise pass into the target cell. The ROS may be produced in the cytosol of target cells, or within one or more organelles within the target cell. All possibilities are encompassed within the scope of the invention. Most suitably, however, the ROS is produced within the target cell(s), which helps to reduce the possibility of side-reactions with non-target (e.g. healthy) cells.

Singlet O₂ (like other ROS) interacts with other molecules in essentially two ways: it can react chemically with them or transfer its excitation energy to them returning to the ground state while the other molecule enters an excited state. These phenomena are usually classified as trapping and quenching of ¹Δg O₂ respectively & Gutteridge, Free radicals in biology and medicine, Oxford University Press, 2007). Singlet oxygen traps include an abundant group of chemical compounds which can serve as model objects for initial assessment of the oxygenation rate, and proof-of-principle, imitating, to some extent, mechanisms of the biological effects caused by photo-oxygenation. Therefore implementation of these compounds enables evaluation of the efficiency of direct photo-excitation of molecular oxygen under desired conditions. This may enable the most advantageous “therapeutic action window” to be determined, i.e. the radiation wavelengths at which the target tissue constituents have maximum absorption, or undesirable tissues (e.g. those surrounding the target tissue or cells (such as skin layers) have minimal absorption. This may beneficially allow the radiation wavelength and intensity of radiation used in accordance with the methods of the invention to be selected according to the necessary tissue penetration depth and desired biostimulative effect.

### Therapeutic Applications

The apparatus and methods of the invention can be used to treat one or more diseases and disorders in an animal, and preferably in a human.

The therapeutic methods of the invention may be particularly suitable for the treatment of diseases, conditions and/or infections that can be targeted (and treated) through the destruction and death (e.g. by apoptosis) of infected or unhealthy cells. Most advantageously, the diseases, conditions and disorders are treatable using non-invasive methods of the invention, i.e. that do not require concurrent administration of active agents and/or penetration of the body by surgery. For instance, it is beneficial to be able to treat the target cells or tissues through the skin of the subject, e.g. by penetration of light energy through the skin. However, in addition to in vivo treatments and methods, ex vivo and in vitro methods for the production of ROS and/or singlet oxygen (¹Δ_{g}), and the treatment of diseased tissue and/or cells are also encompassed.

Therapeutic uses and applications for the apparatus and methods of the invention include: the treatment of various neoplastic and non-neoplastic diseases and disorders. Neoplastic diseases/cancers and related conditions include breast carcinomas, lung carcinomas, gastric carcinomas, oesophageal carcinomas, oral carcinomas, colorectal carcinomas, liver carcinomas, ovarian carcinomas, thecomas, arrhenoblastomas, cervical carcinomas, endometrial carcinoma, endometrial hyperplasia, endometriosis, fibrosarcomas, choriocarcinoma, head and neck cancer, nasopharyngeal carcinoma, laryngeal carcinomas, hepatoblastoma, Kaposi's sarcoma, melanoma, skin carcinomas, hemangioma, cavernous hemangioma, hemangioblastoma, pancreas carcinomas, retinoblastoma, astrocytoma, glioblastoma, Schwannoma, oligodendroglioma, medulloblastoma, neuroblastomas, rhabdomyosarcoma, osteogenic sarcoma, leiomyosarcomas, urinary tract carcinomas, thyroid carcinomas, Wilm's tumor, renal cell carcinoma, prostate carcinoma, abnormal vascular proliferation associated with phakomatoses, edema—such as that associated with brain tumors, and Meigs' syndrome. Non-neoplastic conditions include infections and inflammatory conditions, such as meningitis, encephalitis, abscess, cerebral malaria, rheumatoid arthritis, psoriasis, atherosclerosis, diabetic and other proliferative retinopathies, chronic inflammation and lung inflammation. Other therapeutic uses for the molecules and compositions of the invention include the treatment of microbial infections and associated conditions, for example, bacterial, viral, fungal or parasitic infection.

Preferably the methods of the invention are carried out by applying/delivering the required radiation (light energy) topically to the skin of an individual; to cells within the skin (e.g. dermis); or to subcutaneous cells or tissues, by penetration of the radiation through the skin layers. Where skin/tissue penetration is desired, the wavelength and/or intensity of the radiation may be adjusted to provide the desired depth of penetration and dosage level. Alternatively, however, when deep/internal organs, tissues or cells are to be targeted, the radiation may be delivered beneath the skin (e.g. inside the body), such as by using fibre optic devices to deliver light energy or other means of delivering the desired radiation directly to internal body structures.

The methods of the invention may be performed in isolation, or in conjunction with additional therapeutic agents that may enhance or expedite the death of target cells and tissues. For example, such additional therapeutic agents may be chemotherapeutic agents or antimicrobial agents. For example, it is envisaged that the methods of the invention may provide synergistic effects with chemotherapy for the treatment of cancer or antimicrobial (e.g. antiviral) agents, by initiating, causing or promoting the death of target cells.

Whilst the present preferred embodiments of the semiconductor laser emit laser light in a continuous wave regime, a pulsed operation regime may also be used, and such alternative embodiments fall within the scope of the present invention.

Furthermore, whilst the herein described preferred embodiments comprise a quantum dot semiconductor diode laser, it is envisaged that other types of laser emitting optical radiation having a substantially similar bandwidth profile may equally be used in the herein described methods of activating ROS and/or singlet oxygen (¹Δ_{g}), and PDT.

Equally, it is envisaged that semiconductor laser structures comprising nitrogen based gain mediums, configured to emit optical radiation having substantially similar bandwidth profiles as the herein described quantum dot semiconductor diode laser may also be used with the methods of the present invention.

The herein described embodiments are for illustrative purposes only, and are not intended to be limiting.

The invention will now be further illustrated by way of the following non-limiting examples.

### Experimental Analyses and Examples

Unless otherwise indicated, commercially available reagents and standard techniques in molecular biological and biochemistry were used. General purpose reagents were purchased from Sigma-Aldrich Ltd (Poole, Dorset, UK).

To exemplify the applicability of the PDT methods and apparatuses of the invention for the treatment of target cells, the effect of NIR irradiation on target cells and tissues was assessed. The Examples demonstrate that a semiconductor laser diode of the invention is able to photoactivate oxygen radicals present within and/or in proximity of the target cells to cause disturbances in cellular processes and ultimately cell death.

### Near Infrared Quantum Dot Laser

A fibre coupled InGaAs/InAs quantum dot laser diode (QD LD; Innolume GmbH) (NA=0.22, fibre core diameter=105 μm, laser diode chip length=3 mm) in continuous wave regime was used as an irradiation source. The QD gain structure was grown on an n+ GaAs (100) substrate by molecular beam epitaxy. The laser active region consisted of five planes of self-organised quantum dots separated by 33 nm thick GaAs spacer layers. Each QD plane was formed by deposition of 2.5 mono-layers of InAs followed by a 5.5 nm thick InGaAs quantum well. The density of quantum dots per layer was approximately 5×10¹⁰ cm⁻² and the sizes were estimated to be 5×12×12 nm. N- and P-type Al_{0.8}Ga_{0.2} As cladding layers were doped with Si and Be respectively with a carrier concentration of 5×10¹⁷ cm⁻³. The active region was placed in the centre of a short-period Al_{0.3}Ga_{0.7} As (2 nm)/GaAs (2 nm) superlattice which acts as a waveguiding layer. The total thickness of the laser waveguide was about 0.35 μm. The structure was terminated by a 0.6 μm thick GaAs contact layer that was heavily doped with Be. This had an emission spectrum centred at a wavelength of between 1260 and 1270 nm. Its spectral bandwidth was estimated to be about 12 nm full width at half maximum (FWHM). Maximal output power was measured to be approximately 1.3 W with an injection current of 5 A at a QD LD operation temperature of 30° C.

The use of this laser source allowed selective targeting of the strongest near infrared absorption band of molecular oxygen, attributed to the direct transitions from the ground state to the lowest excited state O₂ (¹Δ_{g})←O₂ (³Σ_{g}⁻), at power levels which are commonly used in biological experiments.

### Cell Cultures

Briefly, HeLa cells and HaCaT immortalised keratinocytes were cultured in Dulbecco's modified Eagle's medium (DMEM) supplemented with 10% fetal bovine serum (FBS) at a temperature of 37° C. in a 4.5% of CO₂ atmosphere as previously described (Buth et al., (2007), Europ. J. Cell. Biol. 86, pp 747-761) and were grown to confluence. Prior to test measurements, the cells were washed with phosphate-buffered saline (PBS) and incubated with a 0.1% trypsin/EDTA for 3 to 15 min to encourage disaggregation. Cells were then transferred to modified phosphate-free Tyrode's medium (TM), 140 mM NaCl, 3.6 mM KCl, 1.2 mM MgCl₂, 1.8 mM CaCl₂, and 10 mM HEPES, pH 7.4 for further use in fluorescence experiments.

### Experimental Set Up and Temperature Control

The fluorescence experiments were conducted on a Zeiss Axio Observer A1 inverted fluorescent microscope with Axopatch 200B amplifier (Molecular Devices), and a QD LD radiation source coupled to an optic fibre and optic filter emitting irradiation centred between 1260 and 1270 nm. The measuring chamber was in fluidly connected to an ISNA-34 peristaltic pump (IsmaTec, Swiss) to keep cells in the chamber constantly perfused with TM at a rate of approximately 1 ml/min.

To monitor the temperature of the media surrounding the cells, a thermocouple connected via a TC-08 Thermocouple Data Logger to a PC with PicoLog software (Pico Technology) was mounted in the chamber.

To avoid overheating of living cells during laser irradiation the reaction/measurement chamber was constantly perfused with TM solution. As controls (described below) heating was monitored during 3 min of irradiation at approximately 1270 nm and approximately 830 nm. 830 nm was chosen as a negative control, because this wavelength does not activate singlet oxygen production. The results (not shown) demonstrated that during the period of laser irradiation the temperature of the media inside the measurement chamber gradually and continuously rose, returning to RT after the laser had been deactivated. The maximum temperature reached during the 3 min irradiation was approximately 25.5° C. at highest radiation dose of approximately 119.4 J/cm². Less than one degree of difference in temperature increase was observed for the experiments using a wavelength of approximately 1270 nm compared to the wavelength of approximately 830 nm. This data thus demonstrated that the continuous perfusion system was able to adequately regulated cell temperature during the irradiation assays. By contrast, in a control experiment in which the continuous perfusion system was switched off, the temperature increase was found to be dependent on the volume of solution (media) in the chamber: and at a 2 mm depth of media the temperature rise was approximately 6° C. above RT during the 3 min assay.

### Example 1

### Measurements of Singlet Oxygen in Anoxia Air-Saturated Solution

Trap of singlet oxygen 98% naphthacene (Sigma-Aldrich) was used as a substrate for photo-oxygenation by means of direct laser excitation of molecular oxygen (Hallwell & Gutteridge, Free radicals in biology and medicine, Oxford Uni. Press, 2007; Zolotovskaya et al., (2009) ECBO'09, Munich, Germany, ThB4; Krasnovsky et al., (2003) Biochem., 68, pp 1178-1182; and Krasnovsky & Ambartsumian (2004) Chem. Phys. Lett. 400: pp 531-535). The rate constant of singlet O₂ reactions with naphthacene was determined to be approximately 10⁶ M⁻¹s⁻¹. Since naphthacene is not a water-soluble compound, carbon tetrachloride (CCl₄, Fluka) was chosen as a solvent medium.

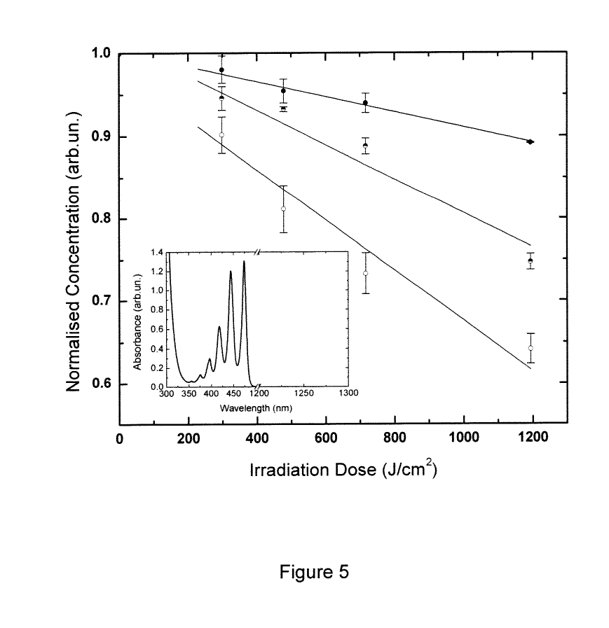

The absorption spectrum of naphthacene is shown in FIG. 5 (inset). In CCl₄, the main absorption maxima of naphthacene is located at approximately 474 nm. It should be noted that naphthacene demonstrates an absence of any resolvable absorption in the vicinity of 1270 nm. The interaction of this compound with singlet O₂ is known to be purely chemical, accompanied by formation of endoperoxides and loss of absorbance in the visible spectral range (Gadd & Clayman (1972) Cel. Mol. Life. Sciences. 28: pp 719-720). The molar absorption coefficient, corresponding to the principal absorption maxima, was estimated to be approximately 1.25×10⁴ M⁻¹ cm⁻¹ (Hallwell & Gutteridge, 2007; Zolotovskaya, 2009).

The concentration of the ¹O₂ trap in solution used for the assessment of the singlet oxygen production did not exceed 200 μM in order to avoid possible concentration quenching in the solution. Air-saturated solutions with a volume of 3 ml were irradiated in 10 mm rectangular quartz cuvettes whilst undergoing agitation. The absorption spectra of the solutions under investigation were measured before and after irradiation, using a PerkinElmer Lambda 900 UVNIS/NIR spectrometer. The difference in absorption at a given wavelength before and after laser irradiation was used as a measure of the ¹O₂ formation ratio. A control sample was used as a measure of photobleaching under normal room lighting conditions and responses were normalised accordingly. The dose dependent ¹O₂ formation ratio measurements were carried out at an incident to the quartz cuvette at a power of up to 1 W at a fixed irradiation time of 10 min for DPBF and 20 min for naphthacene at atmospheric pressure and room temperature (25° C.). A collimated laser beam with a diameter of 8 mm was used for irradiation. The QD LD irradiation centred between 1260 and 1270 nm led to appreciable bleaching of the air-saturated solutions. The QD LD irradiation centred at approximately 1270 nm led to appreciable bleaching of the air-saturated solutions, and the concentration of naphthacene was found to reduce linearly in accordance with the increase in radiation dose (as depicted in FIG. 5). In oxygen-free solutions, the results obtained after a 30 min zero-grade nitrogen purification demonstrated a decrease in photobleaching for both solutions of ¹ Δ_{g} O₂ traps in the examined dose range. An increase in the photoreaction rates was attained for the 20 min zero-grade oxygen purified solutions. This subsequently led to two fold increases in photobleaching of naphthacene at an irradiation dose of 1200 J/cm². These data indicate that the increase in photoreaction rates was directly proportional to the increase in oxygen concentration.

The experimental results suggest that the observed oxygenation of the singlet O₂ trap in organic solution, as determined by reactive ¹Δ_{g} O₂ production, was a result of direct laser excitation at a wavelength of between 1260 and 1270 nm, and indicates that similar photo-oxygenation reactions may be achieved in living cells.

### Assessment of Singlet Oxygen Activation by Fluorescence Analyses

Fluorescence analyses of living cells by means of exogenous reporter molecules represent the vast majority of spectroscopic analyses performed on cells. A large number of exogenous fluorophores has been created and can be used for a variety of different purposes and applications in the field of biological monitoring, ranging from cellular function control; using fluorescent reporter dyes or molecules for various biochemical species, e.g. Ca²⁺, Mg²⁺, pH, nucleic acid sequences, etc.; to demarcation of tumours (Vo-Dinh, ed., Biomedicalphotonics, CRC Press, 2003). The optical sensor-based analysis using ion-selective fluorophores, allows for accurate determination and quantification of cellular ionic environment, providing spatial and temporal information on targeted-ion fluctuations, which could be associated with various pathological conditions. In Examples 2 and 3 we use fluorescence analysis to measure intracellular calcium level, [Ca²⁺]ᵢ, and oxidative stress in various cell types in response to laser-induced ROS formation from oxygen species by means of direct activation.

Many methods are available to identify reactive species, and some of them have become widely used. Photoluminescence of various fluorescent ‘probes’, for example, often increases in response to oxidative stress at elevated O₂ concentration or introduction of ROS-generating drugs. Dihydro-compounds are representative of traditional fluorescence probes which may be used to detect ROS production in view of the relative ease with which they are oxidised (Halliwell & Gutteridge, Free radicals in biology and medicine, Oxford University Press, 2007). Provided such compounds are cell-permeable (i.e. capable of passing through intact, healthy cell membranes), they provide a highly effective means of monitoring oxidative stress within living cells.

In Examples 2 and 3, dihydroethidium was used as the fluorescence compound due to its excellent cellular retention, low level of toxicity to living cells, and the relatively low cellular trauma that may be caused during the process of entering living cells. It also possesses desirable fluorescence/staining properties. In this regard, dihydroethidium is readily taken up by living cells and exhibits a blue fluorescence in the cytoplasm when viewed in UV light. However, once oxidised within a living cell the resultant dihydroxyethidium molecule intercalates the bases of DNA and exhibits red fluorescence. Furthermore, the signal is relatively stable, because dihydroxyethidium is well retained inside the cell by virtue of its cationic nature, which makes it unable to cross a healthy cell membrane in order to penetrate into or escape from a living cell.

### Example 2

### Oxidative Stress Assay

This Example demonstrates laser-induced oxidative stress and the production of ROS on a HaCaT immortalised skin keratinocyte cell line.

Cells of the spontaneously immortalised skin keratinocyte line, HaCaT (Boukamp et al., (1988), J. Cell. Biol. 106, pp 761-771), were used in the fluorescence assay. HaCaT immortalised skin keratinocytes closely resemble normal keratinocytes in their growth and differentiation characteristics, both in culture and in surface transplants. This cell line retains remarkable ability for normal differentiation even after multiple passages (>140) with stable genetic balance over extended culture periods, without shifting to the tumorigenic phenotype.

Cells were cultured for use as described above. After disaggregation, cells were centrifuged at 1200 rpm for 2 min at 4° C., washed and re-suspended in bath solution and temporarily stored on ice.

The cells were loaded with dihydroethidium, DHE (5 μM; Sigma) in modified Tyrode's medium (TM) for 10 min at 37° C. After incubation cells were washed twice with TM. All fluorescence measurements were carried out in TM solution and different cell types were irradiated with the same dose (intensity, frequency and duration) of NIR radiation, unless otherwise stated.

As a positive control in the absence of laser irradiation sodium hypochlorite, NaOCl (100 μM, 3 min application), was used as a source of ROS. As a negative control the experiment was performed in the presence of α-tocopherol (10 μM, 3 min incubation), which is an effective general quencher of ROS, e.g. singlet oxygen.

Prior to every measurement, cells were transferred into the measurement chamber where they were attached to a at the coverslip bottom of the chamber, and maintained at room temperature (RT) of approximately 23° C. for 20 min.

A microscopic ratiometric fluorescence imaging system was used to monitor the fluorescence intensity for oxidised dihydroethidium at a wavelength of 540 nm using an excitation wavelength of 470 nm. To perform real-time imaging, the light from a xenon lamp (HBO 100) was rapidly directed through the narrowband filters which were alternated in the light pathway. Subsequently, the fluorescence signal at 540 nm was filtered and registered with a 694×520 resolution CCD camera (AxioCam MRm, CarlZeiss) controlled by AxioVision 4.8.4 software (Carl Zeiss) at a 2×2 binning every 5 s for the duration of the experiment. Binning was used to reduce the effects of noise and increase the rate of image acquisition. The image analysis routines of AxioVision software (Carl Zeiss) were used for image and data analysis. The emitted fluorescence intensities at 540 nm for the excitation wavelength was evaluated as an average over at least 20 to 30 cells for each test and control group. Fluorescent data of all cells obtained were averaged and plotted against time with Origin 8.0.

Laser irradiation was carried out with an incident power on the measurement chamber of 0.05, 0.15, 0.2, 0.3 and 0.5 W with a slightly elliptical laser beam spot of about 8×12 mm. An irradiation time of 3 min and an irradiation dose of 47.7 J/cm² at a wavelength of approximately 1260 to 1270 nm were used.

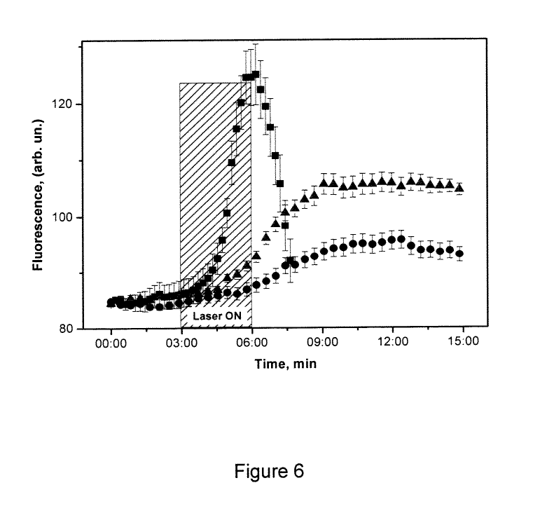

The results of the three experiments performed on HaCaT cells are shown in the graph of FIG. 6. All fluorescence traces demonstrate a clear increase in ROS production within the HaCaT cells, although in each case distinctly different characteristic behaviours are apparent. Notably, the positive control group (in the absence of laser irradiation) exhibited a rapid increase in the dihydroxyethidium (DHOE) fluorescence signal (as a result of increased ROS), which was followed by a subsequent rapid decrease in signal when the oxidizing reagent, NaOCl was then washed out from the measurement chamber (see curve of black squares). By contrast, the fluorescence signals observed after laser irradiation demonstrated an increase in ROS production, which continued even 3 min after the termination of the laser application (see curve of light grey triangles). Interestingly, a marked reduction in fluorescence intensity was observed for the negative control group, HaCaT, which had been pre-incubated for 10 mins in 10 μM α-tocopherol (curves of dark grey circles). This result indicates that the lased-induced intracellular ROS formation can be inhibited quite effectively in the presence of the ROS quencher α-tocopherol; and this suggests that the photo-oxidative effect of laser irradiation at the experimental wavelength is to activate singlet oxygen inside or in the vicinity of the target cells A limited increase in fluorescence intensity for the negative control group was observed, and this might be attributed to auto-oxygenation of the fluorescence compound (Rota et al., (1999), Free Radical Biol. Med. 27, pp 873-881; and Afzal et al., (2003), Biochem. Biophys. Res. Commun. 304, pp 619-624).

A characteristic increase in the ROS fluorescence signal could be observed in all experiments after the termination of laser irradiation. This data thus suggests the possibility that laser irradiation (at a central wavelength of approximately 1260 to 1270 nm and an irradiation dose of 47.7 J/cm²)—may induce or activates a free-radical chain reaction in HaCaT keratinocytes.

It should be noted, therefore, that this fluorescence assay provides qualitative proof of intracellular ROS production (or ROS production in the medium surrounded the test cells) in response to laser irradiation at 1260 to 1270 nm. More probably, the data and experimental results indicate that laser irradiation is capable of directly activating singlet oxygen. However, it may be desirable to use still further singlet oxygen specific fluorescence compounds (Soh (2006) Anal. Bioanal. Chem. 386, pp 532-543) to further elucidate the mechanism of direct photoactivation of singlet oxygen in living cells.

### Example 3

### Oxidative Stress Assay

This Example demonstrates laser-induced oxidative stress and the production of ROS inside a number of different cell types, namely, a HaCaT immortalised skin keratinocyte cell line, HeLa tumour cells and primary keratinocytes. By using primary keratinocytes in addition of cancer cells lines any differences between the reactions of normal and cancer cells lines to irradiation treatment and ROS attack might be seen.

Cell Culture: HeLa cells, and HaCaT immortalized keratinocytes were cultivated at 37° C. in a 4.5% of CO₂ atmosphere as described by Gadd & Clayman (1972), Cel. Mol. Life. Sciences. 28: pp 719-720. The culturing of HaCaT cells is described in more detail above. Primary skin keratinocytes were cultured as described by Kovacs et al. (2009) J. Dermatological Sci., 54(2): pp 106-113. Prior to all experiments the cells were disaggregated with a 0.1% tyrosine/EDTA for 3 to 15 min, transferred to Tyrode's medium (TM) and stored on ice.

Laser photoactivation of molecular oxygen in living cells: In each experiment, test cells were pre-loaded with 5 μM dihydroethidium (DHE) and the level of ROS (e.g. singlet oxygen) formed by laser irradiation was measured. The measurement chamber was constantly perfused during the course of the experiment with room temperature TM at rate of 1 ml/min so as to prevent the cells from over-heating. A laser diode according to the invention was used to irradiate the cell cultures with NIR radiation having a central wavelength of approximately 1260 to 1270 nm.

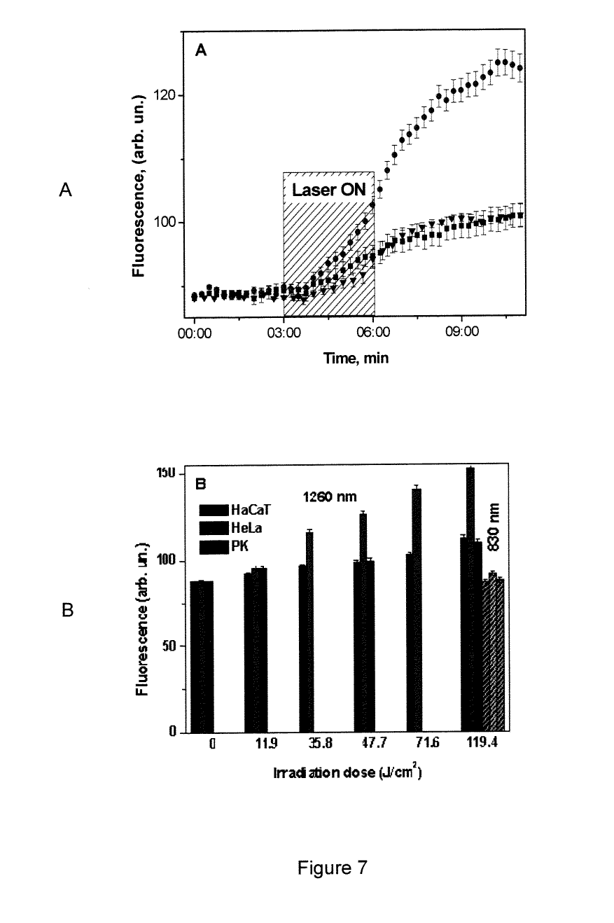

The results of irradiating HaCaT cells with NIR at 47.7 J/cm² are clearly displayed in FIG. 6 (Example 2). To test the effect of NIR radiation on normal and cancer cells similar experiments were conducted to those of Example 2, but using HaCaT cells, primary keratinocytes and HeLa cancer cells, respectively. FIG. 7A shows a comparison between the DHOE fluorescence observed on irradiated HaCaT cells, primary keratinocytes, and HeLa cancer cells, for a laser having a central wavelength of approximately 1260 to 1270 nm and an intensity of approximately 47.7 J/cm². As shown in FIG. 7A the NIR semiconductor laser of the invention irreversibly activates dihydroethidium bromide fluorescence in all cell types, with the most dramatic effects being observed in respect of HeLa cancer cells. The apparent high sensitivity of HeLa cells to NIR irradiation may be a result of their malignant origin or a weaker free radical defence system in comparison to the less diseased/abnormal cell lines tested. This may be an indication that cancerous (i.e. diseased) cells may be more susceptible to photo-oxidative damage by irradiation according to the methods of the invention.

A further experiment was conducted to test the effects of using different irradiation dosages. Therefore, a laser diode according to the invention was used to irradiate these cell cultures with NIR radiation at a range of different intensities, i.e.: 11.9, 35.8, 47.7, 71.6 and 119.4 J/cm²; and the measured DHOE fluorescence results are shown in FIG. 7B. As illustrated, NIR laser-induced dihydroethidium bromide fluorescence appears to demonstrate relatively strong dose dependency without reaching saturation. The increase in effect is especially apparent for HeLa cells (“HeLa”, light grey bars, middle of each column). Meanwhile, the responses of primary keratinocytes (“PK”, dark grey bars, left of each column) and HaCaT keratinocyte cells (“HaCaT”, dark grey bars, left of each column) were essentially the same as each other (showing an increase in proportion to the radiation dose) at each radiation dose tested.

To ensure that the measured effects attributed to cell irradiation were not simply due to (over-) heating of the cells by the laser, a control was carried out in which a laser diode emitting radiation at 830 nm (thus unable to activate singlet oxygen) was employed to provide a comparable level of heating of the TM cell medium as the semiconductor laser at approximately 1260 to 1270 nm used for the test experiments. Since the 830 nm irradiation cannot be absorbed by ¹O₂, it has no effect on ROS-dependent fluorescence in any of the cell types. The results of this control experiment (illustrated in the far right-hand group of columns in FIG. 6B) indicate that the limited amount of heating due to laser irradiation was not responsible for the increase in DHOE fluorescence. Thus, the results provide further support for the hypothesis that irradiation at approximately 1260 to 1270 nm (rather than at 830 nm), induces ROS production through singlet oxygen photoactivation rather than through heat.

A particularly interesting observation was the continued increase in ROS-dependent fluorescence inside the cells even after the irradiation had ceased. This result suggests that, once triggered by irradiation at approximately 1260 to 1270 nm, singlet oxygen might then cause an unexplained chain reaction, leading to further ROS production within the cell, which may eventually trigger apoptosis (see below).

The above-described experimental results suggest that the semiconductor laser of the invention can be used to emit optical radiation having a wavelength centred around approximately 1260 to 1270 nm, and that it may be used to directly photoactivate singlet oxygen radicals in vivo. Furthermore, the results suggest that the intensity of radiation used can be selected according to requirements, e.g. to strike a balance between effectiveness while avoiding undesirable cellular damage. Radiation dosages of at least 10 J/cm² may be used, and a preferred range may be in the region of approximately 50 J/cm² (such as approximately 47.7 J/cm²).

### Example 4

### Intracellular ‘Free’ Calcium Monitoring

This Example demonstrates laser-induced oxidative stress and the disturbance of cellular balance, e.g. ion concentration regulation, inside the different cell types (HaCaT, HeLa and primary keratinocytes) tested in Example 2.

The measurement of the ‘free’ calcium, [Ca²⁺]ᵢ, in the cytoplasm of living cells using calcium-sensitive fluorescent dyes is a well established technique in the art (see for example Moore et al., (1990), Cell Calcium. 11, pp 157-179). For [Ca²⁺]ᵢ fluorescence measurements, dual-excitation ratiometric calcium dyes, have several advantages. One benefit is their reduced sensitivity to artefacts from uneven fluorophore distribution, photobleaching and path length. Another benefit is that [Ca²⁺]ᵢ fluorescence imaging studies require a single intensified camera at the detection site with a rotating filter wheel, containing appropriate bandpass filters to obtain temporally resolved fluorescence measurements (Leybaert et al., (1998), Biophis. J. 75, pp 2025-2029).

In this assay a membrane-permeable radiometric Ca²⁺ indicator, Fura-2 AM (Sigma), was used to quantify intracellular calcium elevation. During cell loading, Fura-2 AM crosses cell membrane and once inside the cell, the acetoxymethyl groups are removed by cellular esterases. Removal of the acetoxymethyl esters regenerates the pentacarboxylate calcium indicator Fura-2 that rapidly binds to Ca²⁺ in a ratio of 1:1, with a concomitant shift in its peak absorbance to a shorter wavelength (Grynkiewicz et al., (1985), J. Biol. Chem., 260, pp 3440-3450).

Test cells used for the Ca²⁺ fluorescence analysis were cultured as described in the preceding Examples. All measurements were performed on the cells reached confluence.

To image [Ca²⁺]ᵢ level before, during and after laser irradiation, the HaCaT and HeLa cells were loaded with Fura-2 AM (5 μM) in modified Tyrode's medium (TM) which contained: 137 mM NaCl, 5.4 mM KCl, 0.5 mM MgCl₂, 5 mM glucose, 10 mM HEPES, 1.8 mM CaCl₂, adjusted to a final pH of 7.4 with NaOH and to 307 mOsm/kg with D-sorbitol. The cells were allowed to incubate in the loading solution at a temperature of 37° C. and in complete darkness for 60 to 90 min in order to maximize the Fura-2 AM de-esterification necessary for Ca²⁺ binding. After loading, the cells were washed twice with the same solution as was used during the loading. The loaded cells were then placed in TM and kept on ice.

A microscopic ratiometric fluorescence imaging system was employed to obtain the ratio of corresponding emitted fluorescence intensities at a wavelength of 510 nm from the Fura-2 utilizing two different excitation wavelengths of 340 and 380 nm, which corresponded to bound and unbound Fura-2 respectively. To perform realtime imaging, the light from a xenon lamp (HBO 100) was rapidly directed through the narrowband filters which were alternated in the light pathway. Subsequently, the fluorescence signal at 510 nm was filtered and registered with a 694×520 resolution CCD camera (AxioCam MRm, CarlZeiss) at a 2×2 binning every 5 s for the duration of the experiment, controlled by AxioVision 4.8.3 software (Carl Zeiss). Binning was used to reduce the effects of noise and increase the rate of image acquisition.

The image analysis routines of AxioVision software (Carl Zeiss) were used for image and data analysis. The emitted fluorescence intensities at 510 nm for both excitation wavelengths of 340 and 380 nm (fem₃₄₀, fem₃₈₀) in each cell were determined from manually segmented regions of the cell in the image (data not shown). The background-corrected fluorescence intensities (fembc₃₄₀, fembc₃₈₀) were attained by subtracting the background, obtained by segmenting a region not containing cells. The fluorescence ratio was then acquired as Ratio (f₃₄₀/f₃₈₀)=fembc₃₄₀/fembc₃₈₀ and subsequently averaged on a number of segmented regions of the HaCaT cells, by at least 20 cells for each measured group in the microscope field-of-view. The laser irradiation conditions were kept the same as for the patch-clamp experiments described in Example 5 below.

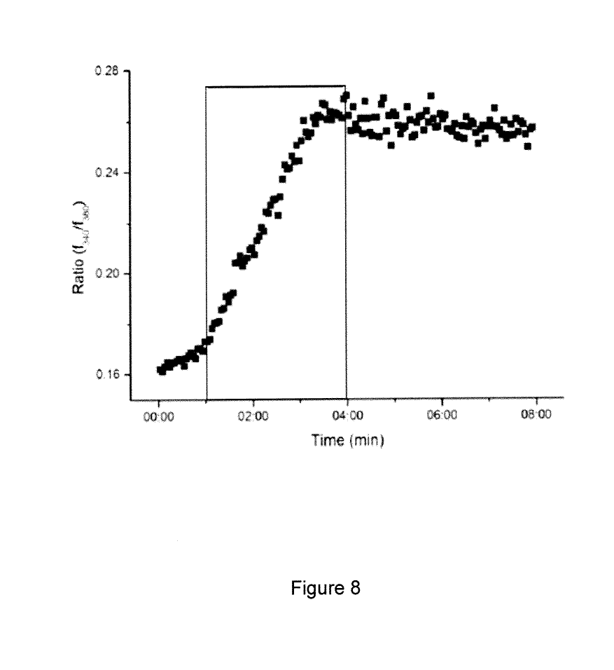

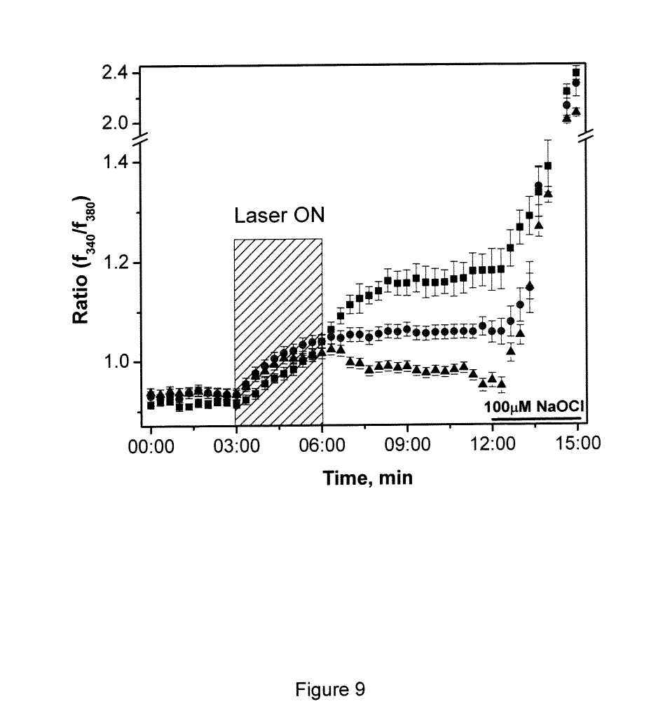

FIGS. 8 and 9 show the intracellular ‘free’ [Ca²⁺]ᵢ fluorescence registered before, after and during 3 min laser irradiation at a central wavelength of approximately 1260 to 1270 nm or 830 nm and an irradiation dose of approximately 47.7 J/cm². Cell responses included an apparent overall increase in the fluorescence ratio, registered immediately after the laser application, with a continuous increase in [Ca²⁺]ᵢ concentration over the 3 min irradiation time. As shown in FIG. 8, the measured fluorescence ratio level in irradiated HaCaT cells increased by more than 1.5 times from the initial value of approximately 0.165±0.017 before irradiation to approximately 0.26±0.03 after about 2.5 min of laser irradiation. Subsequently a plateau in the [Ca²⁺]ᵢ fluorescence was reached and, notably, the ratio value of [Ca²⁺]ᵢ cell response failed to return to the initial equilibrium during a continuous 4 min monitoring after cessation of the laser application.

The results of similar fluorescence experiments are shown in FIG. 9 for HaCaT cells (grey squares) and HeLa cells (dark grey circles), irradiation at approximately 1260 nm at a dose of approximately 47.7 J/cm², in comparison to a negative control irradiation dose of 47.7 J/cm² at approximately 830 nm (in HaCaT cells, black triangles). In these experiments, the fluorescence ratio level increased by more than 1.2 times for HeLa and HaCaT cells during the course of the radiation treatment; specifically, from an initial value of approximately 0.942±0.013 before irradiation to approximately 1.08±0.02 after 3 min of laser irradiation for HeLa cells, and 0.91±0.009 before irradiation to 1.156±0.034 immediately after irradiation for HaCaT cells, Following the cessation of irradiation, intracellular calcium concentration was measured for at least 7 mins and was found to continue to rise. Furthermore, approximately 6 mins after the irradiation treatment had stopped, a large and rapid rise in intracellular calcium was observed, which might suggest the effects of cell death.

This appears to suggest that the intracellular ‘free’ calcium concentration had, as a result of the irradiation treatment, reached a level at which it could not be readily regulated by the intracellular organelles.

It is well known that oxidative stress deregulates Ca²⁺ homeostasis, causing a rise in the intracellular ‘free’ calcium. This can contribute to pro-proliferative effects at low-level as well as to cytotoxic effects at high levels of oxidative stress, for example, perhaps resulting in damage to the endoplasmic reticulum Ca²⁺ uptake system and potentially interfering with Ca²⁺ efflux through plasma membranes (Halliwell & Gutteridge, Free radicals in biology and medicine, Oxford University Press, 2007).

Thus, this Example demonstrates laser-induced cytosolic free calcium changes, which may also have dramatic effects on normal cellular metabolism. The results suggest that the observed increase in cytoplasmic Ca²⁺ concentration and ROS production is likely to be associated with direct molecular oxygen photoactivation.

### Example 5

### Laser-Induced Ion Channel Activation in HaCaT Keratinocytes

The free oxygen concentration within the structures of living cells is known to be significantly less than that in air-saturated solutions, due to the activity of the respiration system, which functions as a source of energy, and a natural biochemical mechanism that decreases formation of the reactive oxygen species. This process is thought to reduce the probability of direct photo-excitation of free oxygen in true biological systems. Nevertheless it cannot entirely exclude such reactions due to the high concentration of bound oxygen. As has been previously reported, direct singlet oxygen dosimetry in living cell cultures is additionally complicated by short ¹Δ_{g} O₂ lifetime (Jarvi et al., (2006), Photochem. Photobiol. 82, pp 1198-1210), restricting applicable ¹O₂ registration techniques to the detection of secondary messengers or other types of cell response on singlet oxygen formation.

Cell signalling occurs in various ways via specific receptors on the receiving cells and subsequent generation of a transmembrane signal. The signal that actually initiates the cellular response of the cell is the flux of ions across the membrane. The membrane proteins that give rise to selective ion permeability are called ion channels. Some of these channels are able to sense the electrical potential across the membrane. Such voltage-gated channels open or close in response to the magnitude of the membrane potential, allowing the membrane permeability to be regulated by the potential. Some ion channels, however, are gated by extracellular chemical signals, others by intracellular signals such as second messengers, and still others respond to mechanical stimuli or temperature changes. Many types of ion channels have now been identified at the molecular level, and this diversity generates a wide spectrum of electrical characteristics (Hille, Ionic channels of excitable membranes, Sinauer Associates Inc., 1984). The extracellular patch clamp method provides an extremely sensitive means to measure ionic currents flowing through cell membranes, which first allowed the detection of single-channel currents in biological membranes (Neher (1991), Nobel Prize Lecture; and Sakmann, (1991), Nobel Prize Lecture). In this Example, we use the patch clamp technique to monitoring the voltage-gated single-channel activity in HaCaT immortalised skin keratinocytes in response to laser irradiation having a central wavelength of approximately 1260 to 1270 nm.

Again, HaCaT cells were chosen as a model object for this assay due to their unique differentiation capacity and preserved sensitivity to environmental signals. As previously noted, HaCaT immortalised skin keratinocytes closely resemble normal keratinocytes in their growth and differentiation characteristics, both in culture and in surface transplants. This cell line retains remarkable ability for normal differentiation even after multiple passages (>140) with stable genetic balance over extended culture periods, without shifting to the tumorigenic phenotype. For this experimental assay, HaCaT keratinocytes were cultured as described in Example 1, i.e. in Dulbecco's modified Eagle's medium (DMEM) supplemented with 10% fetal bovine serum (FBS) at a temperature of 37° C. in a 4.5% of CO₂ atmosphere and were grown to confluence. Prior to test measurements, the cells were washed with phosphate-buffered saline (PBS) and incubated with a 0.1% trypsin/EDTA for 15 min to encourage disaggregation, then centrifuged at 200 g for 2 min at 4° C., washed and re-suspended in bath solution and temporarily stored on ice. The bath solution for the electrophysiological measurements was constituted of: 120 mM NaCl, 10 mM CaCl₂, 2 mM MgCl₂, 5 mM D-glucose, 10 mM HEPES, adjusted to pH 7.3 with NaOH, and adjusted to final 300 mOsm/kg with D-sorbidol. The microelectrode solution had the same chemical composition as the bath solution.

The ion channel activity in HaCaT cells were monitored at an incident power on a measurement chamber of 0.2 W with a slightly elliptical laser beam spot of about 8×12 mm and an irradiation time of 3 min. The single-channel ion currents were recorded in cell-attached configuration, filtered with 2 kHz low-pass Bessel filter and sampled at 10 kHz using an Axopatch 200B patch-clamp amplifier, Digidata 1440 A converter and pCLAMP 10 software (Molecular Devices, USA). The electrophysiological measurements were carried out after formation of the pipette-membrane seals with resistances of 2 to 4 GΩ, measured by applying a 0.1 mV voltage pulse in the pipette and monitoring the resulting current flow. The quiescent activity in HaCaT cells exhibited an rms current of about 0.2 pA. It should be mentioned that the high resistance of a ‘giga-seal’ reduces the background noise of the recording by an order of magnitude (Hamill et al., (1988), PfliigersArch. 391, pp 85-100). The single-channel events were constantly recorded at a holding potential of −100 mV during 2 min before and 3 min after laser irradiation. The recording chamber, with a volume of 0.3 ml was continuously perfused at a rate of 1 ml/min with room temperature bath solution (23° C.) in order to maintain an adequate temperature control.

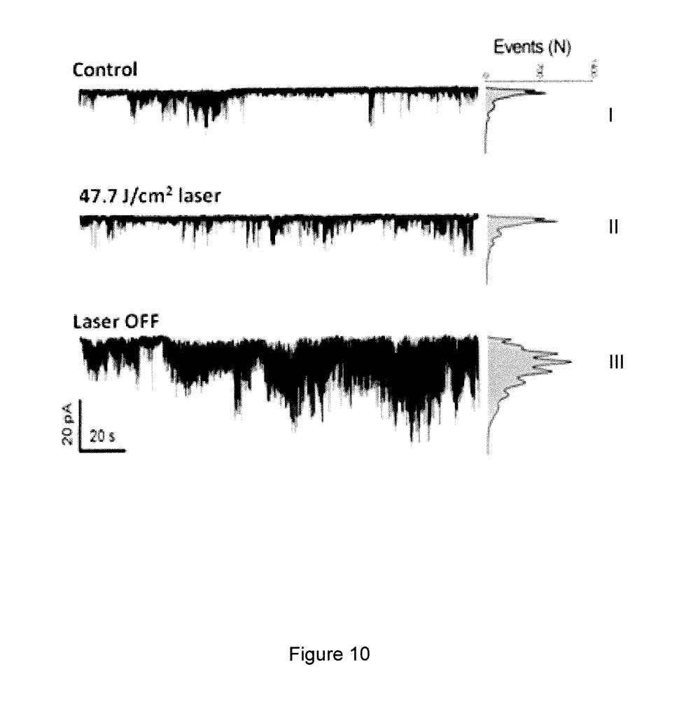

FIG. 10 shows representative traces of single-channel ion currents in HaCaT keratinocytes recorded at a laser irradiation dose of approximately 47.7 J/cm². An analysis of single-channel activity for the control measurements acquired before laser irradiation demonstrated a distribution of the current amplitudes between −5 and −25 pA with a dwell open time of around 1 to 3 ms (see trace I in FIG. 10). This characteristic behaviour is typically associated with low-voltage activated calcium (Ca²⁺) channels within non-excited tissue (Kostyuk (1999), Neuroscience 92, pp 1157-1163). The tests showed that neither amplitude (dwell open time) nor the number of open channels significantly changed during 3 min laser irradiation for the experiments using incident dose of 47.7 J/cm². An immediate response after laser application was registered, demonstrating a rise in open events of more than an order of magnitude with no changes in dwell open time.

Open channel event statistics for an irradiation dose of approximately 47.7 J/cm² are demonstrated in FIG. 10, right-hand segment, in which histogram I illustrates a single-channel event before laser irradiation, II illustrates the single-channel event during laser irradiation, and III illustrates the single channel event after the full irradiation treatment at a dose of approximately 47.7 J/cm².

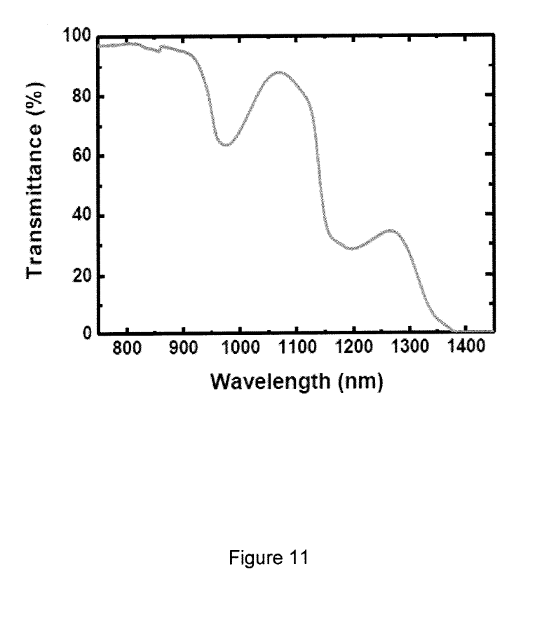

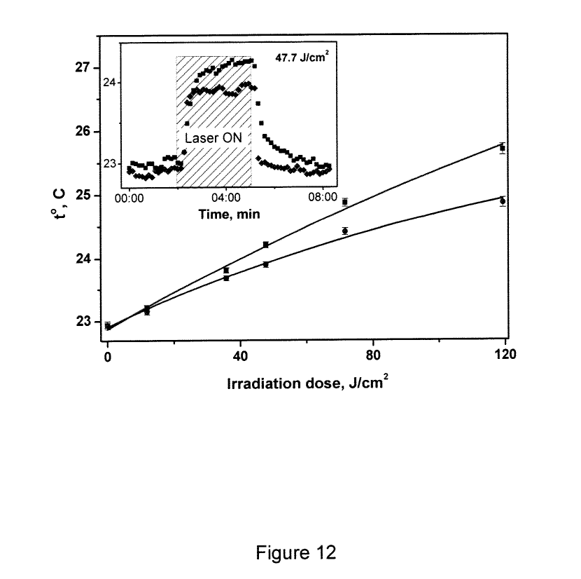

The transmission spectrum of the bath solution that was used in the experiments was also measured and the results are presented in FIG. 11. The electrophysiological medium had a transmittance of about 34% at a wavelength of approximately 1260 to 1270 nm, which might lead to a temperature increase in the assay medium and, thus, possible activation of the observed ion channels. Therefore, to ensure against this possibility leading to the observed results, temperature measurements in the recording chamber at evaluated incident power levels were taken using a thermocouple deepen into bath solution was connected through analogue-digital signal convertor TC-08 Thermocouple Data Logger to PC with PicoLog software (Pico Technology). The results of this assay (see FIG. 12), demonstrated that any heating of the electrophysiological bath solution had a maximum increase of approximately 2° C., at an incident maximal dose of 119.4 J/cm² after 3 min irradiation time for both wavelengths (i.e. approximately 1260 to 1270 nm and approximately 830 nm). This study also demonstrated that the temperature in the electrophysilogical chamber began to rise when laser irradiation was commenced, but began to return to room temperature immediately after termination of irradiation.

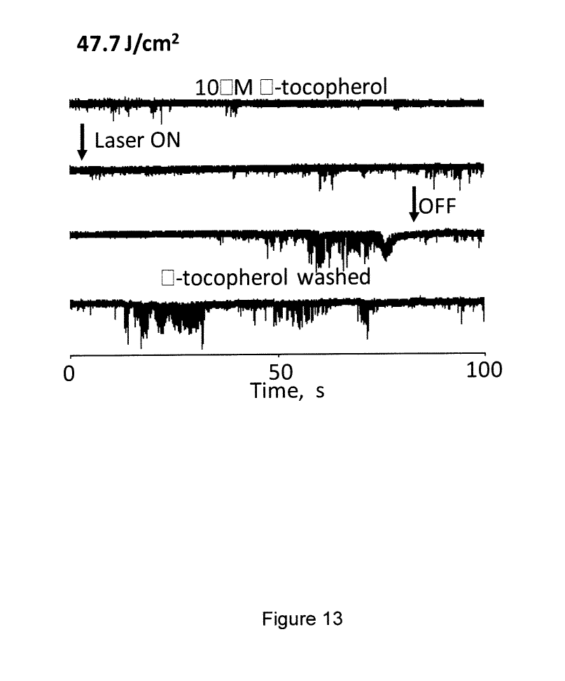

As a control for laser-induced ion channel activation, control experiments were carried out in the presence of the specific ROS quencher α-tocopherol (10 mM, 10 min pre-incubation) at an irradiation dose of approximately 47.7 J/cm², and the results are shown in FIG. 13. Recorded ion channel responses for the control experiments demonstrated a significant decrease in the number of open events compared to previously described results in the absence of α-tocopherol, as compared to the results shown in FIG. 10). These results also suggest that the observed low-voltage Ca²⁺ channel activation in HaCaT keratinocytes at incident dose of approximately 47.7 J/cm² can be attributed to the singlet oxygen formation occurring as a response to photo-excitation at a central wavelength of approximately 1260 to 1270 nm. The negative control experiments were planned in order to investigate the ion channel activity for the laser excitation at a wavelength of 830 nm, which is shifted from the molecular oxygen absorption bands and coincides well with a transparency window of the bath solution, as shown in FIG. 11. The observed temperature change in the cell medium is shown in FIG. 12, and indicates that temperature increase is unlikely to be a relevant factor in these tests.

Cytosolic Ca²⁺ has been recognised as ubiquitous second messenger and Ca²⁺ channels as an essential part for important signalling responses in various cell types. They account entirely for the regenerative electrical excitability in muscles of mollusc, nematode, etc. In some other preparations they can be demonstrated to coexist with Na⁺ channels and to make a partial contribution to electrical excitability in smooth and cardiac muscle and in the nerve cell bodies. They are also found in all secretory gland cells and nerve terminals, where they regulate secretion (Hagiwara & Byerly (1981), Anna. Rev. Neurosci. 4, pp 69-125; Kostyuk, et al., (1982), J. Mernb. Biol. 70, pp 171-179; and Tsien (1983), Annu. Rev, Physiol. 45, pp 341-358). As a broad generalisation, all excitable cells translate their electricity into action by Ca²⁺ fluxes modulated by voltage-sensitive Ca²⁺ channels. Calcium ions are intracellular messengers capable of activating many cell functions, serving as the only link to transduce depolarisation into all the non-electrical activities controlled by excitation. As in non-excitable cells, and in keratinocytes in particular, Ca²⁺ channel activity and the concomitant change in membrane potential may influence their growth and differentiation properties, as well as their viability (Csernoch, et al., (2000), Exp. Dermatol. 9, pp 200-205; Dascalu et al., (2000), Investig. Dermatol. 115, pp 714-718; and Koegel & Alzheimer (2001), FASEB J. 15, pp 145-154). Normally, in a resting cell, the cytoplasmic ‘free’ calcium level is very low at around 0.1 μM or less, which is maintained by the combined action of a Na⁺/Ca²⁺ exchange system on the surface membrane and ATP-dependent pumps on mitochondria and other intracellular organelles. Therefore, Ca²⁺ ion influx and an increased in intracellular free calcium concentration can serve as a qualitative measure of physiological activity and as a signal to mobilise the protein-synthetic machinery of the cell in response to a variety of intra- and extracellular stimuli (Halliwell & Gutteridge, Free radicals in biology and medicine, Oxford University Press, 2007). It can also, clearly, significantly disrupt the normal activities of the affected cells.

In summary, the ion channel activation in HaCaT keratinocytes was observed as a response to photoactivation in the near infrared spectral range. The characteristic behaviour that was observed was associated with low-voltage activated Ca²⁺ channels. The control experiment, conducted with the singlet oxygen quencher α-tocopherol, demonstrated a significant inhibition of the laser-induced channel activity. Thus, the results demonstrate that the single channel activation on the HaCaT plasma membrane can be attributed to the laser-induced production of singlet oxygen in the cell. As indicated above, this activity may lead to a variety of cellular responses and ultimately to cell death.

### Example 6

### Assessment of Laser-Induced HeLa Cell Death

To assess whether direct activation of ROS by laser irradiation at approximately 1260 to 1270 nm is capable of causing (tumour) cell death, a cell death assay was carried out. The cell death assay was carried out on HeLa cells using the CytoTox kit (Promega) according to the manufacturers instructions. HeLa cells were seeded in 96 well plate format and a SpectraMax 250 Elisa platereader (Molecular Devices) was used for measurements. HeLa cells in the test were irradiated at radiation doses of approximately 47.7 and 119.4 J/cm². As a negative control the cells were irradiated at approximately 830 nm (as previously described), which causes a comparable temperature increase in the measurement chamber, but does not activate singlet oxygen. As a positive control cells were treated with BI2536, which is a PLK1 inhibitor that induces tumour cell death.

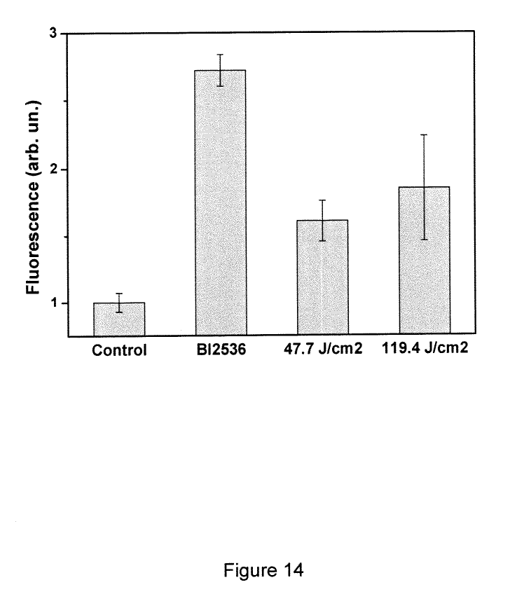

The results of the assay are presented in FIG. 14, and clearly show that both irradiation doses cause a significant level of cell death (above the negative control). The higher radiation dose causing, as broadly expected, a greater level of cell death. Thus, the data demonstrate a significant increase in cell death rate within the population of HeLa cells irradiated with either test dose. In the positive control group the highest death rate was found. Parallel counting of detached cells, which tends to be the preliminary step in the chain of cell apoptotic events (and indicates the first signs of apoptosis), evidenced an increasing number of HeLa cells in the growth medium after 3 mins of irradiation at approximately 1260 to 1270 nm at either radiation dose of 47.7 and 119.4 J/cm².

In conclusion, these experiments clearly demonstrate that the ROS production can be activated in living organisms and cells by direct irradiation at appropriate wavelengths; and that such ROS activation (particularly singlet oxygen), can trigger cellular imbalances, such as increases in cytosolic calcium concentration and, ultimately, cell death. Thus, these results show the potential efficacy of laser irradiation for the treatment (e.g. reduction and/or irradication) of cancers in living organisms, such as animals and humans. Other diseases that can be treated by apoptosis or necrosis of affected cells, and infections, may also be treated using the apparatuses and methods of the invention.

Although particular embodiments of the invention have been disclosed herein in detail, this has been done by way of example and for the purposes of illustration only. The aforementioned embodiments are not intended to be limiting with respect to the scope of the appended claims, which follow. It is contemplated by the inventors that various substitutions, alterations, and modifications may be made to the invention without departing from the spirit and scope of the invention as defined by the claims.

1. A semiconductor laser diode comprising:
an active region having a gain medium comprising one or more InGaAs/InAs quantum dot layers; and wherein
the laser diode is arranged in operation to emit laser light having a central wavelength within the spectral range of approximately 1250 to 1280 nm.

2. The laser diode of claim 1, wherein each InGaAs/InAs quantum dot layer comprises:
one InGaAs layer equal to one mono-layer;
one InAs layer equal to two and a half mono-layers; and
the InGaAs and InAs layers are arranged contiguous with each other; and wherein one or more quantum dots (QD) are comprised in the InAs layer.

3. The laser diode of claim 2, further comprising a GaAs spacer layer arranged to separate adjacent InGaAs/InAs layers, and arranged contiguous with the InAs layer defining a contact surface for facilitating the formation of an array of quantum dots in the InAs layer.

4. The laser diode of claim 3, wherein the density of the quantum dots formed in the InAs layer is within the range of approximately 5×10¹⁰ cm to 6×10¹¹ cm² per contact surface.

5. The laser diode of claim 1, wherein the number of layers of the InGaAs/InAs gain medium is at least three.

6. The laser diode of claim 1, wherein the number of layers of the InGaAs/InAs gain medium is at least five.

7. The laser diode of claim 1, wherein the number of layers of the InGaAs/InAs gain medium is less than or equal to fifteen.

8. The laser diode of claim 1, wherein the full width at half maximum (FWHM) spectral bandwidth of the emitted laser light is less than approximately 50 nm.

9. The laser diode of claim 1, wherein the FWHM spectral bandwidth of the emitted laser light is less than approximately 25 nm.

10. The laser diode of claim 1, wherein the FWHM spectral bandwidth of the emitted laser light is approximately 12 nm.

11. The laser diode of claim 1, wherein the laser diode emits continuous wave laser light, or a pulsed laser light.

12. A method of directly forming a reactive oxygen species (ROS), the method comprising:
exposing a medium comprising a potential source of ROS to a semiconductor laser diode, the semiconductor laser diode configured to emit laser light having a central wavelength within the spectral range of approximately 1250 to 1280 nm.

13. The method of claim 12, wherein the medium is substantially free of photosensitising agents that promote the formation of ROS.

14. The method of claim 12, wherein the medium comprises molecular oxygen.

15. The method of claim 14, wherein the ROS is singlet oxygen.

16. The method of claim 12, wherein the semiconductor laser diode comprises an active region having a gain medium comprising one or more InGaAs/InAs quantum dot layers.

17. The method of claim 16, wherein the semiconductor laser diode comprises two or more InGaAs/InAs quantum dot layers and a GaAs spacer layer arranged to separate adjacent InGaAs/InAs layers.

18. A method for treating cancer in an individual, the method comprising:
exposing at least one tumour cell to laser light emitted from a semiconductor quantum dot laser diode at an intensity and for a period of time sufficient to generate reactive oxygen species (ROS) within or in the vicinity of the at least one tumour cell, whereby the death of one or more tumour cells occurs, and wherein the method does not involve the use of photosensitising agents that promote the formation of ROS within or in the vicinity of the at least one tumour cell.

19. The method of claim 18, wherein singlet oxygen is generated from molecular oxygen within or in the vicinity of the one or more tumour cells, and wherein the emitted laser light has a central wavelength within the spectral range of approximately 1250 to 1280 nm.

20. The method of claim 18, wherein the semiconductor laser diode comprises two or more InGaAs/InAs quantum dot layers and a GaAs spacer layer arranged to separate adjacent InGaAs/InAs layers.
